(19) United States
(12) Patent Application Publication
(10) Pub. No.: US 2025/0027098 A1
(43) Pub. Date: Jan. 23, 2025

(54) METHOD FOR SELECTING TARGET SITES FOR SITE-SPECIFIC GENOME MODIFICATION IN PLANTS

(71) Applicant: Monsanto Technology LLC, St. Louis, MO (US)

(72) Inventors: Brent BROWER-TOLAND, St. Louis, MO (US); Paul S. CHOMET, St. Louis, MO (US); Robert T. GAETA, Chesterfield, MO (US); Andrei Y. KOURANOV, Chesterfield, MO (US); Jonathan C. LAMB, Wildwood, MO (US); Richard J. LAWRENCE, Kirkwood, MO (US); Ruth WAGNER, Chesterfield, MO (US)

(73) Assignee: Monsanto Technology LLC, St. Louis, MO (US)

(21) Appl. No.: 18/908,523

(22) Filed: Oct. 7, 2024

Related U.S. Application Data
(63) Continuation of application No. 16/338,335, filed on Mar. 29, 2019.
(60) Provisional application No. 62/402,724, filed on Sep. 30, 2016.

(51) Int. Cl. C12N 15/82

(52) U.S. Cl. CPC C12N 15/8213; C12N 15/8261

(57) ABSTRACT

The present disclosure provides methods and compositions for identification of optimal genomic loci in plant genome for site-directed integration in plants.

### CROSS-REFERENCE TO RELATED APPLICATION

This application is a continuation of U.S. application Ser. No. 16/338,335, filed internationally on Sep. 29, 2017, which is a national stage application under 35 U.S.C. § 371 of International Application No. PCT/U52017/054378, filed on Sep. 29, 2017, which claims priority to U.S. Provisional Application No. 62/402,724, filed on Sep. 30, 2016,each of which is incorporated herein by reference in its entirety.

### REFERENCE TO AN ELECTRON SEQUENCE LISTING FIELD

The contents of the electronic sequence listing (777052057701SEQLIST.xml; Size: 1,289,294 bytes; and Date of Creation: Oct. 3, 2024) is herein incorporated by reference in its entirety.

### FIELD

The present disclosure provides methods for selecting target sites for site-specific genome modification in plant genomes.

### BACKGROUND

Site-specific genome modification in plant genomes provides a means to develop plants with specific traits and to facilitate plant breeding programs. For the development of new agronomic traits, site-specific genome modification enzymes are used for site-specific genome editing, and for site-specific targeted integration of a DNA of interest. Site-specific transgene integration in a plant genome provides significant improvement over random integration of a transgene in the development of new traits.

There is a need to develop methods for identification of target sites for site-specific genome modification in a plant genome. The present disclosure describes a target site selection process to identify genomic regions that are suitable as target sites of site-specific genome modification enzymes. The process includes bioinformatics analysis of intron and exon gene structure, non-coding RNA sequence, small RNA sequence, sequence redundancy and chromatin modification consensus sequence sites. Additional information is agronomic data tied to haplotype windows to guide selection of specific target sites for integration of DNA of interest in a given plant. Site-specific integration of DNA of interest will reduce development costs and increase optimal agronomic trait development in the site-specific modified plant genome.

### BRIEF SUMMARY

Several embodiments relate to a recombinant sequence comprising a non-genic plant genomic sequence and a DNA of interest. In some embodiments, the DNA of interest is integrated into a target site in the non-genic plant genomic sequence. In some embodiments, the target site is located in a haplotype window associated with a neutral to positive impact on one or more agronomic traits. In some embodiments, the target site is further located at genetic distance greater than 1 cM of a haplotype window that is associated with a negative impact on one or more agronomic traits. In some embodiments, the target site is located within a small genomic region (less than 1000 bp) of low genetic diversity, where the low genetic diversity is defined as having from one to ten distinguishable haplotypes across all germplasm in the intended heterotic group, the intended maturity group, or the intended heterotic and maturity group. In some embodiments, the haplotype window is based on physical distance. In some embodiments, the physical distance comprises between 40 base pairs and the full length of the chromosome, with at least 99% sequence similarity across the targeted germplasm and contains two or fewer indels of transposon size (˜3 kb). In some embodiments, the haplotype window is defined by genetic distance. In some embodiments, the genetic distance is 0.1 cM, 0.5 cM, 1 cM, 2 cM, 3 cM, 4 cM, or 5 cM. In some embodiments, the agronomic trait is one or more selected from the group consisting of: yield, car relative maturity, car height, car number, increased car size, grain moisture, increased car dry weight per plant, increased number of kernels per ear, increased weight per kernel, increased number of kernels per plant, decreased car void, extended grain fill period, test weight, pod number, number of seed per pod, pod position on the plant, number of internodes, incidence of pod shatter, grain size, decreased days from planting to maturity, increased stalk size, increased number of leaves, increased plant height growth rate in vegetative stage, plant architecture, resistance to lodging, percent seed germination, seedling vigor, juvenile traits, efficiency of germination (including germination in stressed conditions), growth rate (including growth rate in stressed conditions), increased number of root branches, increased total root length, efficiency of nodulation and nitrogen fixation, enhanced nitrogen use efficiency, increased water use efficiency as compared to a control plant, efficiency of nutrient assimilation, resistance to biotic and abiotic stress, carbon assimilation, physiology, enhanced disease or pest resistance, or environmental or chemical tolerance, enhanced cold tolerance, nutritional enhancement, enhanced seed protein, enhanced seed starch, enhanced seed oil, plant height, enhanced plant morphology, growth and development, and stay green rating. In some embodiments, the non-genic plant genomic sequence is a corn genomic sequence or a soybean genomic sequence. In some embodiments, the corn genomic sequence is selected from the group consisting of SEQ ID NOs: 123-172, 294, 299-551, 555 and 556. In some embodiments, the corn genomic sequence is a B Chromosome sequence selected from the group consisting of SEQ ID NO:300-551. In some embodiments, the soybean genomic sequence is selected from the group consisting of SEQ ID NOs: 251-282, 554. In some embodiments, the target site comprises at least 75, at least 80, at least 90, at least 100, at least 200, at least 300, at least 400, at least 500, at least 600, at least 700, at least 800, at least 900, or at least 1000 nucleotides. In some embodiments, the DNA of interest comprises a gene expression cassette comprising a sequence selected from an insecticidal resistance gene, a herbicide tolerance gene, a nitrogen use efficiency gene, a water use efficiency gene, a nutritional quality gene, a DNA binding gene, a selectable marker gene, a target site for a site-specific genome modification enzyme, a recombinase target site, and any combination thereof. In some embodiments, the target site comprises one or more of the criteria selected from the group consisting of: (i) the target site is located greater than 2 kb from a 5′ or a 3′ end of a gene in the plant genome; (ii) the target site is located more than 1 kb from a 5′ or a 3′ end of a repeat region in the plant genome, and wherein the repeat region is at least 2 kb in length; (iii) the target site is located more than 1 kb from a 5′ or a 3′ end of a repressive chromatin mark in the plant genome; (iv) the target site is located more than 200 bases from a small RNA (sRNA) hotspot in the plant genome, and wherein the sRNA hotspot is a sequence from 0.2 to 1 kb in length; (v) the target site is within a region of the plant genome of low DNA methylation; (vi) the target site is not within a region of the plant genome associated with at least one DNA methylation read containing an MspJi motif or a LpnPI motif; or (vii) the target site is within a region of the plant genome that exhibits a total k-mer redundancy score of less than or equal to 30%. In some embodiments, the target site comprises one or more of the criteria selected from the group consisting of: (i) the target site is located greater than 2 kb from a 5′ or a 3′ end of a gene in the plant genome; (ii) the target site is located more than 1 kb from a 5′ or a 3′ end of a repeat region in the plant genome, and wherein the repeat region is at least 2 kb in length; (iii) the target site is located more than 1 kb from a 5′ or a 3′ end of a repressive chromatin mark in the plant genome; (iv) the target site is located more than 200 bases from a small RNA (sRNA) hotspot in the plant genome, and wherein the sRNA hotspot is a sequence from 0.2 to 1 kb in length; (v) the target site is within a 100 bp, 150 bp, 200 bp, 250 bp, 300 bp, 350 bp, 400 bp, 500 bp, 550 bp, 600 bp, 650 bp, 700 bp, 750 bp, 800 bp, 850 bp, 900 bp, 950 bp, or 1,000 bp region of the plant genome of low DNA methylation; (vi) the target site is not within a 100 bp, 150 bp, 200 bp, 250 bp, 300 bp, 350 bp, 400 bp, 500 bp, 550 bp, 600 bp, 650 bp, 700 bp, 750 bp, 800 bp, 850 bp, 900 bp, 950 bp, or 1,000 bp region of the plant genome associated with at least one DNA methylation read containing an MspJi motif or a LpnPI motif; or (vii) the target site is within a 100 bp, 150 bp, 200 bp, 250 bp, 300 bp, 350 bp, 400 bp, 500 bp, 550 bp, 600 bp, 650 bp, 700 bp, 750 bp, 800 bp, 850 bp, 900 bp, 950 bp, or 1,000 bp region of the plant genome that exhibits a total redundancy score of less than or equal to 35%, 34%, 33%, 32%, 31%, 30%, 25%, 20%, 15%, 10% or 5%. In some embodiments, the recombinant nucleic acid is present in a plant, plant cell, or plant part.

Several embodiments relate to a method of making a transgenic plant cell comprising a DNA of interest targeted to at least one non-genic plant genomic sequence, the method comprising: (i) selecting a target site located within a haplotype window associated with a neutral to positive impact on one or more agronomic traits; (ii) introducing a site-specific genome modification enzyme into a plant cell, wherein the site-specific genome modification enzyme cleaves the target site in the non-genic plant genomic sequence; (iii) introducing a DNA of interest; (iv) targeting the DNA of interest to the target site, wherein the cleavage of the target site facilitates integration of the DNA of interest into the non-genic plant genomic sequence; and (v) selecting transgenic cells comprising the DNA of interest integrated into the non-genic plant genomic sequence. In some embodiments, the method of making a transgenic plant cell comprising a DNA of interest targeted to at least one non-genic plant genomic sequence the method comprising: (i) selecting a target site located within a haplotype window associated with a neutral to positive impact on one or more agronomic traits and where the target site is located at a genetic distance of greater than 10 cM of a haplotype window that is associated with a negative impact on one or more agronomic traits; (ii) introducing a site-specific genome modification enzyme into a plant cell, wherein the site-specific genome modification enzyme cleaves the target site in the non-genic plant genomic sequence; (iii) introducing a DNA of interest; (iv) targeting the DNA of interest to the target site, wherein the cleavage of the target site facilitates integration of the DNA of interest into the non-genic plant genomic sequence; and (v) selecting transgenic cells comprising the DNA of interest integrated into the non-genic plant genomic sequence. In some embodiments, the method of making a transgenic plant cell comprising a DNA of interest targeted to at least one non-genic plant genomic sequence the method comprising: (i) selecting a target site located within a haplotype window associated with a neutral to positive impact on one or more agronomic traits and where the target site is located at a genetic distance of greater than 10 cM of a haplotype window that is associated with a negative impact on one or more agronomic traits; (ii) selecting a haplotype window where the genetic distance is 0.1 cM, 0.5 CM, 1 cM, 2 CM, 3 cM, 4 cM, or 5 cM; (iii) introducing a site-specific genome modification enzyme into a plant cell, wherein the site-specific genome modification enzyme cleaves the target site in the non-genic plant genomic sequence; (iv) introducing a DNA of interest; (v) targeting the DNA of interest to the target site, wherein the cleavage of the target site facilitates integration of the DNA of interest into the non-genic plant genomic sequence; and (vi) selecting transgenic cells comprising the DNA of interest integrated into the non-genic plant genomic sequence. In some embodiments, the agronomic trait is one or more selected from the group consisting of: yield, car relative maturity, car height, car number, increased car size, grain moisture, increased car dry weight per plant, increased number of kernels per ear, increased weight per kernel, increased number of kernels per plant, decreased ear void, extended grain fill period, test weight, pod number, number of seed per pod, pod position on the plant, number of internodes, incidence of pod shatter, grain size, decreased days from planting to maturity, increased stalk size, increased number of leaves, increased plant height growth rate in vegetative stage, plant architecture, resistance to lodging, percent seed germination, seedling vigor, juvenile traits, efficiency of germination (including germination in stressed conditions), growth rate (including growth rate in stressed conditions), increased number of root branches, increased total root length, efficiency of nodulation and nitrogen fixation, enhanced nitrogen use efficiency, increased water use efficiency as compared to a control plant, efficiency of nutrient assimilation, resistance to biotic and abiotic stress, carbon assimilation, physiology, enhanced disease or pest resistance, or environmental or chemical tolerance, enhanced cold tolerance, nutritional enhancement, enhanced seed protein, enhanced seed starch, enhanced seed oil, plant height, enhanced plant morphology, growth and development, and stay green rating. In some embodiments, the non-genic plant sequence is a soybean genomic sequence or a corn genomic sequence. In some embodiments, the corn genomic sequence is selected from the group consisting of SEQ ID NOs 123-172, 294, 299-551, 555 and 556. In some embodiments, the corn genomic sequence is a B Chromosome sequence selected from the group consisting of SEQ ID NO:300-551. In some embodiments, the soybean genomic sequence is selected from the group consisting of SEQ ID NOs: 251-282. In some embodiments, the target site comprises one or more of the criteria selected from the group consisting of: (i) the target site is located greater than 2 kb from a 5′ or a 3′ end of a gene in the plant genome; (ii) the target site is located more than 1 kb from a 5′ or a 3′ end of a repeat region in the plant genome, and wherein the repeat region is at least 2 kb in length; (iii) the target site is located more than 1 kb from a 5′ or a 3′ end of a repressive chromatin mark in the plant genome; (iv) the target site is located more than 200 bases from a small RNA (sRNA) hotspot in the plant genome, and wherein the sRNA hotspot is a sequence from 0.2 to 1 kb in length; (v) the target site is within a region of the plant genome of low DNA methylation; (vi) the target site is not within a region of the plant genome associated with at least one DNA methylation read containing an MspJi motif or a LpnPI motif; or (vii) the target site is within a region of the plant genome that exhibits a total k-mer redundancy score of less than or equal to 30%. In some embodiments, the target site comprises one or more of the criteria selected from the group consisting of: (i) the target site is located greater than 2 kb from a 5′ or a 3′ end of a gene in the plant genome; (ii) the target site is located more than 1 kb from a 5′ or a 3′ end of a repeat region in the plant genome, and wherein the repeat region is at least 2 kb in length; (iii) the target site is located more than 1 kb from a 5′ or a 3′ end of a repressive chromatin mark in the plant genome; (iv) the target site is located more than 50 bp, 100 bp, 150 bp, 200 bp, 250 bp, 300 bp, from a small RNA (sRNA) hotspot in the plant genome, and wherein the sRNA hotspot is a sequence from 0.2 to 1 kb in length; (v) the target site is within a 50 bp, 100 bp, 150 bp, 200 bp, 250 bp, 300 bp, 350 bp, 400 bp, 500 bp, 550 bp, 600 bp, 650 bp, 700 bp, 750 bp, 800 bp, 850 bp, 900 bp, 950 bp, or 1,000 bp region of the plant genome of low DNA methylation; (vi) the target site is not within a 100 bp, 150 bp, 200 bp, 250 bp, 300 bp, 350 bp, 400 bp, 500 bp, 550 bp, 600 bp, 650 bp, 700 bp, 750 bp, 800 bp, 850 bp, 900 bp, 950 bp, or 1,000 bp region of the plant genome associated with at least one DNA methylation read containing an MspJi motif or a LpnPI motif;

or (vii) the target site is within a 100 bp, 150 bp, 200 bp, 250 bp, 300 bp, 350 bp, 400 bp, 500 bp, 550 bp, 600 bp, 650 bp, 700 bp, 750 bp, 800 bp, 850 bp, 900 bp, 950 bp, or 1,000 bp region of the plant genome that exhibits a redundancy score of less than or equal to 35%, 34%, 33%, 32%, 31%, 30%, 25%, 20%, 15%, 10% or 5%. In some embodiments, the target site comprises at least 75, at least 80, at least 90, at least 100, at least 200, at least 300, at least 400, at least 500, at least 600, at least 700, at least 800, at least 900, at least 1000 nucleotides. In some embodiments, the DNA of interest comprises a gene expression cassette comprising a sequence selected from an insecticidal resistance gene, a herbicide tolerance gene, a nitrogen use efficiency gene, a water use efficiency gene, a nutritional quality gene, a DNA binding gene, a selectable marker gene, and any combination thereof. In some embodiments, the site-specific genome modification enzyme is selected from an endonuclease, a recombinase, a transposase, and any combination thereof. In some embodiments, the endonuclease is selected from a meganuclease, a zinc finger nuclease, a transcription activator-like effector nuclease (TALEN), a Cas9 nuclease, a Cpf1 nuclease, a Cas12a nuclease, a Cas12e nuclease, a CasX nuclease, a Cas 12d nuclease, a CasY nuclease, a Cas12b nuclease, a C2C1 nuclease, a Cas12c nuclease, a C2C3 nuclease, a C2C4 nuclease, a C2C5 nuclease, a C2C6 nuclease, a C2C7 nuclease, a C2C8 nuclease, a C2C9 nuclease, a C2C10 nuclease, a Cas13a nuclease, a Cas13b nuclease, and a Cas13c nuclease. In some embodiments, the recombinase is a tyrosine recombinase attached to a DNA recognition motif, or a serine recombinase attached to a DNA recognition motif. In some embodiments, the tyrosine recombinase attached to a DNA recognition motif is selected from the group consisting of a Cre recombinase, a Flp recombinase, and a Tnp1 recombinase. In some embodiments, the serine recombinase attached to a DNA recognition motif is selected from the group consisting of a PhiC31 integrase, an R4 integrase, and a TP-901 integrase. In some embodiments, the transposase is a DNA transposase attached to a DNA binding domain. In some embodiments, transcription activator-like effector nuclease (TALEN) DNA binding site within the target site of corn genomic sequence is selected from the SEQ ID NOs presented in Table 1. In some embodiments, the transcription activator-like effector nuclease (TALEN) DNA binding site within the target site of soybean genomic sequence is selected from the SEQ ID NOs presented in Table 2. In some embodiments, the DNA of interest is an exogenous sequence. In some embodiments, the DNA of interest comprises one or more transgenes. In some embodiments, the DNA of interest is integrated into the target site via a non-homologous end joining. In some embodiments, the DNA of interest is integrated into the target site via a homologous recombination. In some embodiments, the recombinant nucleic acid is present in a plant, plant cell, or plant part.

### BRIEF DESCRIPTION OF THE DRAWINGS

FIG. 1 is a general work flow diagram illustrating one embodiment of steps in the method of site selection for targeted integration.

FIG. 2 illustrates a screen shot sample of a Genome Browser output for a 10 kb region of chromosome 1 (CR01) of the corn B73 reference genome from position 287440 kb to 287449 kb. Relative redundancy scores (horizontal line marked “Zm.B73Redundancy score”) are illustrated by vertical bars, with the region between 278446 kb and 287449 kb having high redundancy. An exon for the endogenous gene GRMZM2G138382 is illustrated by a gray horizontal bar from 287440 kb to approximately 287442 kb. The horizontal arrow labeled “2 kb” shows the distance to the 5′-end of SEQ ID NO:299. MspJI methylation consensus sites are illustrated by vertical bars on the horizontal line labeled “Methylation by MspJI”. Repeat regions are indicated by horizontal black bars with several positioned between 287287444.5 kb-287449 kb. The horizontal arrow labeled “1 kb” shows the distance to the 3′-end of SEQ ID NO:299. H3K27me3 methylation consensus sequence region is indicated by vertical bars on the horizontal line labeled “H3K27me3 peak”, with a double peak region positioned at 287441.3kb to 287442.3kb. The position of SEQ ID NO: 130 is illustrated by the horizontal line at the top and is positioned approximately from 287442.7 kb to 287445.9 kb. SEQ ID NO:130 is a sequence region about 3.4 kb in length representing at least 4 specific TALEN target sites. A region encompassing a TALEN specific target site, represented by SEQ ID: 294, is within the region represented by SEQ ID NO: 130 and SEQ ID NO:299. The position of SEQ ID NO: 299 is illustrated by the horizontal line at the top and is positioned approximately from 287444 kb to 287445.9 kb. The position of SEQ ID NO: 294 is illustrated by the horizontal line at the bottom and is positioned approximately from 287444 kb to 287445.9 kb. The vertical thick arrow on the horizontal line representing MspJI sites illustrates the position of the TALEN binding sites (SEQ ID NO:35 and SEQ ID NO:94) for the TALEN target site represented by SEQ ID NO:294.

FIG. 3 illustrates an enlarged region of chromosome 1 (CR01) of the corn B73 reference genome from FIG. 2 corresponding to the region of nucleotide 287442700kb to 287226211kb. Additionally, the MspJI DNA methylation profile calculated for this region is plotted as vertical bars, with relative counts of 0 to 6 (Y-axis). The nucleotide region of each of SEQ ID NO:130, SEQ ID NO:299, and SEQ ID NO:294 are indicated by the horizontal double-arrow lines. The nucleotide position selected for TALEN binding sites (SEQ ID NO:35 and SEQ ID NO:94) and TALEN induced double-strand break (DSB) is illustrated by the thick, black horizontal line.

FIG. 4 provides a graph comparing the percent integration of donor polynucleotides into seven sites on the corn genome LH244. Histograms show the percent integration of nucleotides into the corn genome at seven sites, SEQ ID NOs: 32/91, 33/92,34/93, 35/94, 295/296, 297/298, and 304/305 along with the percent integration for the negative controls corresponding to each site. Error bars represent Standard Deviation. Double asterisks (**) identify sites with significantly different (p>0.05) integration frequencies than their negative controls. The panel below indicates DNA methylation status of each targeted region in the genome where “+” indicates methylated and “−” indicates non-methylated regions. The methylated regions were identified by genome-wide MspJI/LpnPI sequencing as described in this application.

FIG. 5 illustrates a 9.6 kb region of chromosome 2 (CR02) of the soy Williams 82 reference genome from position 49329900 kb to 49339882 kb. This region is represented by SEQ ID NO:257. The Y-axis shows both the redundancy scores (k-mer) and the MspJI methylation profile along the length of the sequence (X-axis). The box at position 493353399 kb to 493363399 kb is expanded in FIG. 6, and is the region of TALEN target site selection.

FIG. 6 illustrates a 1 kb nucleotide region (SEQ ID NO:554) of the graph from FIG. 5, from position 49335399 kb to 49336386 kb. At the expanded scale, a region of relative low redundancy score and a relatively low methylation profile is identified as a region for site-specific genome modification (horizontal bar).

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of the present disclosure employs, unless otherwise indicated, conventional techniques of biochemistry, chemistry, molecular biology, microbiology, cell biology, genomics, plant breeding, and biotechnology, which are within the skill of the art. See Green and Sambrook, MOLECULAR CLONING: A LABORATORY MANUAL, 4th edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)); Harlow and Lane, eds. (1988) ANTIBODIES, A LABORATORY MANUAL; ANIMAL CELL CULTURE (R. I. Freshney, ed. (1987)); RECOMBINANT PROTEIN PURIFICATION: PRINCIPLES AND METHODS, 18-1142-75, GE Healthcare Life Sciences; C. N. Stewart, A. Touracv, V. Citovsky, T. Tzfira eds. (2011) Plant Transformation Technologies (Wiley-Blackwell); and R. H. Smith (2013) PLANT TISSUE CULTURE. TECHNIQUES AND EXPERIMENTS (Academic Press, Inc.).

Any references cited herein are incorporated by reference in their entireties.

As used herein, the singular form “a,” “an,” and “the” include plural references unless the context clearly dictates otherwise. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

As used herein, the term “about” indicates that a value includes the inherent variation of error for the method being employed to determine a value, or the variation that exists among experiments.

As used herein, the term “plant” includes a whole plant and any progeny, cell, tissue, or part of a plant. A progeny plant can be from any filial generation, e.g., F₁, F₂, F₃, F₄, F₅, F₆, F₇, etc. The term “plant parts” include any part(s) of a plant, including, for example and without limitation: seed (including mature seed and immature seed); a plant cutting; a plant cell; a plant cell culture; a plant protoplast; a plant organ (e.g., pollen, embryos, flowers, fruits, shoots, leaves, roots, stems, and explants). A plant tissue or plant organ may be a seed, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the donor plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, cars, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

A plant cell is the structural and physiological unit of the plant. Plant cells, as used herein, includes protoplasts and protoplasts with a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant.

As used herein, the term “plant genome” refers to a nuclear genome, a mitochondrial genome, or a plastid (e.g., chloroplast) genome of a plant cell.

As used herein, the term “corn” refers to Zea mays or maize and includes all plant varieties that can be bred with corn, including wild maize species.

As used herein, the term “soybean” refers to Glycine max and includes all plant varieties that can be bred with soybean, including wild soybean species.

As used herein, the term “haplotype” refers to a chromosomal region within a haplotype window defined by at least one polymorphic marker. The unique marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or transgene. Any such change in a haplotype would be included in our definition of what constitutes a haplotype so long as the functional integrity of that genomic region is unchanged or improved.

As used herein, the term “haplotype window” refers to a chromosomal region that is established by statistical analyses known to those of skill in the art and is in linkage disequilibrium. Thus, identity by state between two inbred individuals (or two gametes) at one or more marker loci located within this region is taken as evidence of identity-by-descent of the entire region. Each haplotype window includes at least one polymorphic marker. Haplotype windows are mapped along cach chromosome in the genome.

As used herein, the term “polymorphic marker” refers to a polymorphic nucleic acid sequence or nucleic acid feature. A “polymorphism” is a variation among individuals in sequence, particularly in DNA sequence, or feature, such as a transcriptional profile or methylation pattern. Useful polymorphisms include single nucleotide polymorphisms (SNPs), insertions or deletions in DNA sequence (Indels), simple sequence repeats of DNA sequence (SSRs) a restriction fragment length polymorphism, a haplotype, and a tag SNP. A genetic marker, a gene, a DNA-derived sequence, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation.

In one aspect, a “polymorphic marker” can be a detectable characteristic that can be used to discriminate between heritable differences between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

As used herein, the term “polynucleotide” refers to a nucleic acid molecule containing multiple nucleotides and generally comprises at least 2, at least 5, at least 10, at least 20, at least 30, at least 40, at least 50, at least 100, at least 250, at least 500, at least 1000, at least 1500, at least 2000, at least 2500, at least 3000, at least 5000, or at least 10,000 nucleotide bases. As an example, a polynucleotide provided herein can be a plasmid. A specific polynucleotide of 18-25 nucleotides in length may be referred to as an “oligonucleotide”. Nucleic acid molecules provided herein include deoxyribonucleic acids (DNA) and ribonucleic acids (RNA) and functional analogues thereof, such as complementary DNA (cDNA). Nucleic acid molecules provided herein can be single stranded or double stranded. Nucleic acid molecules comprise the nucleotide bases adenine (A), guanine (G), thymine (T), cytosine (C). Uracil (U) replaces thymine in RNA molecules. The symbol “N” can be used to represent any nucleotide base (e.g., A, G, C, T, or U). The symbol “K” can be used to represent a G or a T/U nucleotide base.

As used herein, the term “genic region” or “genic sequence” refers to a polynucleotide sequence that comprises an open reading frame encoding at least one RNA and/or polypeptide. The genic region may also encompass any identifiable adjacent 5′ and 3′ non-coding nucleotide sequences involved in the regulation of expression of the open reading frame up to about 2 Kb upstream of the coding region and 1 Kb downstream of the coding region, but possibly further upstream or downstream. A genic region further includes any introns that may be present in the genic region. Further, the genic region may comprise a single gene sequence, or multiple gene sequences interspersed with short spans (less than 1 Kb) of non-genic sequences.

As used herein, the term “non-genic plant genomic sequence” or “non-genic plant sequence” or “intergenic sequence” or “intergenic region” refers to a native DNA sequence found in the genome of a plant, devoid of any open reading frames, gene sequences, or gene regulatory sequences. Furthermore, the non-genic sequence does not comprise any intron sequence (specifically, introns are excluded from the definition of non-genic). The non-genic sequence cannot be transcribed or translated into protein.

As used herein, the term “recombination” refers to the exchange of nucleotides between two nucleic acid molecules. The term “homologous recombination” (HR) refers to the exchange of nucleotides at a conserved region shared by two nucleic acid molecules. Homologous recombination HR includes symmetric homologous recombination and asymmetric homologous recombination. Asymmetric homologous recombination can also mean unequal recombination. As used herein, “non-homologous end joining” (NHEJ) refers to the ligation of two ends of double-stranded DNA without the need of a homologous sequence to direct the ligation. Methods for detecting recombination include, but are not limited to, 1) phenotypic screening, 2) molecular marker technologies such as single nucleotide polymorphism-SNP analysis by TaqMan® or Illumina/Infinium technology, 3) Southern blot, 4) PCR, and 4) sequencing.

As used herein, the terms “targeted insertion” and “targeted integration” are used interchangeably.

As used herein, the term “donor sequence” “donor DNA” or “DNA of interest” refers to a nucleic acid/DNA sequence that has been selected for targeted insertion into a host sequence. In one aspect, the host sequence is a plant genomic sequence. A donor sequence can be of any length, for example between 2 and 50,000 nucleotides in length (or any integer value therebetween). In some embodiments, a donor sequence is between about 1,000 and 5,000 nucleotides in length (or any integer value therebetween). In some embodiments, a donor sequence is between about 5,000 and 10,000 nucleotides in length (or any integer value therebetween). In some embodiments, a donor sequence is between about 10,000 and 15,000 nucleotides in length (or any integer value therebetween). In some embodiments, a donor sequence is between about 15,000 and 20,000 nucleotides in length (or any integer value therebetween). In some embodiments, a donor sequence is between about 20,000 and 25,000 nucleotides in length (or any integer value therebetween). In some embodiments, a donor sequence is between about 25,000 and 30,000 nucleotides in length (or any integer value therebetween). In some embodiments, a donor sequence is between about 30,000 and 35,000 nucleotides in length (or any integer value therebetween). In some embodiments, a donor sequence is between about 35,000 and 40,000 nucleotides in length (or any integer value therebetween). In some embodiments, a donor sequence is between about 40,000 and 45,000 nucleotides in length (or any integer value therebetween). In some embodiments, a donor sequence is between about 45,000 and 50,000 nucleotides in length (or any integer value therebetween). A donor sequence may comprise one or more gene expression cassettes that further comprise actively transcribed and/or translated gene sequences. Conversely, the donor sequence may comprise a polynucleotide sequence which does not comprise a functional gene expression cassette or an entire gene (e.g., may simply comprise regulatory sequences such as a promoter), or may not contain any identifiable gene expression elements or any actively transcribed gene sequence. Further, the donor sequence can be DNA or RNA, can be linear or circular, and can be single-stranded or double-stranded. It can be delivered to the cell as naked nucleic acid, as a complex with one or more delivery agents (e.g., liposomes, poloxamers, T-strand encapsulated with proteins, etc.,) or contained in a bacterial or viral delivery vehicle, such as, for example, Agrobacterium tumefaciens or an adenovirus or a Gemini Virus, or a nanovirus, respectively.

As used herein, the term “host sequence” or “host polynucleotide” refers to a polynucleotide sequence in a host plant genome. The term “target site,” as used herein, refers to a polynucleotide sequence that is sufficiently unique in a plant genome to allow targeted genome modification by a site-specific genome modification enzyme. In one aspect, the sequence of the target site is changed from the wild-type sequence, namely the target site is edited. In another aspect, the target site is the site of insertion of a DNA of interest into one specific sequence. In one aspect, the target site is located within a small genomic region (e.g., less than 1500 bp, less than 1000 bp, less than 900 bp, less than 950 bp, less than 850 bp, less than 800 bp, less than 750 bp, less than 700 bp, less than 650 bp, less than 600 bp, less than 550 bp, less than 500 bp, less than 450 bp, less than 400 bp, less than 350 bp, less than 300 bp, less than 250 bp, less than 200 bp, less than 150 bp, less than 100 bp) of low genetic diversity. The term “low genetic diversity” is defined as having from one to ten distinguishable haplotypes across all germplasm in the intended heterotic group, the intended maturity group, or the intended heterotic and maturity group. In some embodiments, the small genomic region comprises 1, 2, 3, 4, 5, 6, 7, 8, 9 or 10 distinguishable haplotypes across all germplasm in the intended heterotic group, the intended maturity group, or the intended heterotic and maturity group.

As used herein, the term “heterotic group” refers to a collection of germplasm which, when crossed to germplasm external to its group (usually another heterotic group), tends to exhibit a higher degree of heterosis (on average) then when crossed to a member of its own group. Two reciprocal heterotic groups define a heterotic pattern. Identification of potential heterotic patterns may be conducted using a population diallele evaluation. The concept of heterotic groups was first developed by maize researchers who observed that inbred lines selected out of certain populations tended to produce superior performing hybrids when hybridized with inbreds from other groups. A heterotic group may also refer to a group of related or unrelated genotypes from the same or different populations, which display similar combining ability when crossed with genotypes from other germplasm groups. Knowledge of the heterotic groups and patterns is helpful in plant breeding. It helps the breeders to utilize their germplasm in a more efficient and consistent manner through exploitation of complementary lines for maximizing the outcome of a hybrid breeding program.

As used herein, the term “maturity group” refers to a classification of some crop varieties based on their growth and development. For example, a soybean with maturity group O or OO only needs a short growing season before harvest; whereas, a soybean with maturity group V and VI needs a longer growing season before the plant is completely developed and ready for harvest. Maturity groups are also described in the context of their indeterminate/determinate growth habit. In corn, relative maturity (RM) group ratings are related to the duration of the growing season, which is related to the growing degree units (GDUs) required by the plant for flowering and reaching physiological maturity. In corn RM groups are listed as early-RM, mid-RM, and late-RM.

As used herein, the term “gene expression cassette” refers to a polynucleotide sequence comprising at least a first polynucleotide sequence capable of initiating transcription of an operably linked second polynucleotide sequence and optionally a transcription termination sequence operably linked to the second polynucleotide sequence. In some aspects, the gene expression cassette may comprise a flanking left homology arm, a right homology arm, or both a left homology arm and a right homology arm.

In some aspects, a sequence of interest provided herein comprises 0, at least 1,at least 2, at least 3, at least 4, at least 5, at least 6, at least 7, at least 8, at least 9, or at least 10 expression cassettes. In some aspects, the sequence of interest provided herein comprises one or more expression cassettes physically and/or operably linked in a cassette stack. In some aspects a sequence of interest comprises an expression cassette adjacent to a left homology arm DNA sequence, a right homology arm DNA sequence, or a left homology arm DNA sequence and a right homology arm DNA sequence. In some aspects, a sequence of interest comprises an expression cassette flanked by homology arm DNA sequences. In some aspects, a sequence of interest comprises an expression cassette that is not flanked by homology arms. In another aspect, a sequence of interest provided herein comprises an endogenous polynucleotide sequence. In some embodiments, the endogenous polynucleotide sequence comprises an intergenic sequence, a native gene, or a mutated gene. In another aspect, a sequence of interest provided herein comprises an exogenous polynucleotide sequence.

In an aspect, a sequence of interest provided herein comprises 0, at least 1, or at least 2 homology arm DNA sequences. When a sequence of interest provided herein comprises at least two homology arm DNA sequences the at least two homology arm DNA sequences can be distinguished by referring to them as a “left homology arm DNA sequence” and a “right homology arm DNA sequence.” In an aspect, a sequence of interest provided herein comprises both a left homology arm DNA sequence and a right homology arm DNA sequence. In an aspect, a right homology arm DNA sequence and a left homology arm DNA sequence provided herein are homologous to a targeted genomic DNA sequence in the plant or plant cell. In an aspect, a right homology arm DNA sequence and a left homology arm DNA sequence are not essentially homologous to each other. In another aspect, a right homology arm DNA sequence and a left homology arm DNA sequence are essentially homologous to each other. In an aspect, a sequence of interest comprises one or more expression cassettes positioned between a right homology arm DNA sequence and a left homology arm DNA sequence. In an aspect, a sequence of interest comprises a sequence for templated genome editing positioned between a right homology arm DNA sequence and a left homology arm DNA sequence. In yet another aspect, at least part of a sequence of interest provided herein is outside of the region comprising a left homology arm DNA sequence, a right homology arm DNA sequence, and one or more cassettes. In another aspect, at least part of a sequence of interest provided herein is within the region comprising a left homology arm DNA sequence, a right homology arm DNA sequence, and a sequence for templated genome editing.

As used herein, the term “homology arm” or “homology arm DNA sequence” refers to a polynucleotide sequence that has at least 80%, at least 85%, at least 90%, at least 91%, at least 92%, at least 93%, at least 94%, at least 95%, at least 96%, at least 97%, at least 98%, at least 99% or 100% sequence identity to a target sequence in a plant or plant cell. A homology arm can comprise at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 250, at least 300, at least 400, at least 500, at least 600, at least 700, at least 800, at least 900, at least 1000, or at least 2500 nucleotides.

In one aspect, the target sequence comprises a protein-coding sequence. In one aspect, the target sequence is a genic sequence. As used herein, a “genic” sequence is a nucleic acid sequence that encodes a protein or a non-protein-coding RNA. A genic sequence can include one or more introns. In another aspect, the target sequence is a non-genic sequence. As used herein, a “non-genic” sequence is a nucleic acid sequence that is not a genic sequence. In another aspect, the target sequence comprises a non-coding sequence. In yet another aspect, the target sequence comprises both a protein-coding sequence and a non-coding sequence. In another aspect, the target sequence does not comprise a gene or a portion of a gene. In some embodiments, the target sequence is linked to a gene of interest. In some embodiments, the target sequence is linked to a transgene integrated in the genome of a plant or plant cell.

In one embodiment, the optimal target site is positioned 2 kb from either the 5′ or the 3′ end of a gene, and the 2 kb genomic region between the target site and the end of the gene is as a region for homologous recombination. As such, the 2 kb sequence is used to engineer homology arms flanking the DNA of interest to be integrated at the target site.

In one embodiment, a target site is selected that is in a region that is greater than 200 nucleotides of a sRNA hotspot.

In one embodiment, a target site is selected that is in a region≤200 nucleotides of a sRNA hotspot. In some embodiments, a target site is selected for integration of a transgene cassette by homologous recombination, wherein the homology arms flanking the transgene cassette are designed such that the transgene cassette integrates at the target site in a ‘head-to-head’ orientation with the sRNA hotspot. This ‘head-to-head’ orientation is where the direction of transcription of the transgene cassette is in the opposite orientation of the direction of transcription of the sRNA hotspot within the genome. This head-to-head orientation will reduce the chance of incorporation of sRNA binding sites during transcription of mRNA from the transgene cassette.

In another embodiment, a target site is selected that is in a region (≤200 nucleotides) of a sRNA hotspot. If the target site is selected for integration of a transgene cassette by homologous recombination, then the homology arms flanking the transgene cassette are designed to have homology to a genomic within the sRNA hotspot, or flanking on the distal 5′-end (for a 5′ homology arm) or the distal 3′-end (for a 3′ homology arm) of the sRNA hotspot. Thereby, and during the HR-dependent integration of the transgene cassette the process of homologous recombination effectively truncates and/or deletes the sRNA hotspot from the final transgenic genomic locus.

As used herein, the term “endogenous sequence” refers to the native form of a polynucleotide, gene, or polypeptide in its natural location in the organism or in the genome of an organism.

As used herein, the term “exogenous sequence” refers to any nucleic acid sequence that has been removed from its native location and inserted into a new location altering the sequences that flank the nucleic acid sequence that has been moved. For example, an exogenous DNA sequence may comprise a sequence from another species, a process referred to as transgenesis. Alternatively, an exogenous DNA sequence may comprise a sequence from the same, or related species, a process referred to as cisgenesis.

As used herein, the term “site-specific genome modification enzyme” refers to any enzyme that can cleave a nucleotide sequence in a site-specific manner. In the present disclosure, site-specific genome modification enzymes include endonucleases, recombinases, transposases, helicases, and any combination thereof. In some embodiments, the site-specific genome modification enzyme is selected from a meganuclease, a zinc finger nuclease, a transcription activator-like effector nuclease (TALEN), a Cas9 nuclease, a Cpf1 nuclease, a Cas 12a nuclease, a Cas12e nuclease, a CasX nuclease, a Cas 12d nuclease, a CasY nuclease, a Cas12b nuclease, a C2C1 nuclease, a Cas 12c nuclease, a C2C3 nuclease, a C2C4 nuclease, a C2C5 nuclease, a C2C6 nuclease, a C2C7 nuclease, a C2C8 nuclease, a C2C9 nuclease, a C2C10 nuclease, a Cas13a nuclease, a Cas13b nuclease, and a Cas 13c nuclease.

As used herein, the terms “homology” and “identity” when used in relation to nucleic acids, describe the degree of similarity between two or more nucleotide sequences. The percentage of “sequence identity” between two sequences is determined by comparing two optimally aligned sequences over a comparison window, such that the portion of the sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. An alignment of two or more sequences may be performed using any suitable computer program. For example, a widely used and accepted computer program for performing sequence alignments is CLUSTALW v1.6 (Thompson, et al. (1994) Nucl. Acids Res., 22:4673-4680). Methods and Compositions for Site-directed Integration in Plants

The present disclosure provides a recombinant sequence comprising a non-genic plant genomic sequence and a DNA of interest, wherein the DNA of interest is inserted into a target site in the non-genic plant genomic sequence, and wherein the target site is located in a haplotype window associated with a neutral to positive impact on one or more agronomic traits, and wherein the target site is further located at genetic distance greater than 1 cM of a haplotype window that is associated with a negative impact on one or more agronomic traits.

The present disclosure also provides a method of making a transgenic plant cell comprising a donor sequence targeted to at least one non-genic plant genomic sequence, the method comprising: (a) selecting a target site located within a haplotype window associated with a neutral to positive impact on one or more agronomic traits; (b) introducing a site-specific genome modification enzyme into a plant cell, wherein the site-specific genome modification enzyme cleaves the target site in the non-genic plant genomic sequence; (c) introducing a donor sequence; (d) targeting the donor sequence to the target site, wherein the cleavage of the target site facilitates integration of the donor sequence into the non-genic plant genomic sequence; and (e) selecting transgenic cells comprising the donor sequence integrated into the non-genic plant genomic sequence.

As used herein, an agronomic trait is a measure of crop performance. Non-limiting examples of agronomic traits, from seeding to harvest, include: yield, car relative maturity, car height, car number, increased car size, grain moisture, increased car dry weight per plant, increased number of kernels per ear, increased weight per kernel, increased number of kernels per plant, decreased car void, extended grain fill period, test weight, pod number, number of seed per pod, pod position on the plant, number of internodes, incidence of pod shatter, grain size, decreased days from planting to maturity, increased stalk size, increased number of leaves, increased plant height growth rate in vegetative stage, plant architecture, resistance to lodging, percent seed germination, seedling vigor, juvenile traits, efficiency of germination (including germination in stressed conditions), growth rate (including growth rate in stressed conditions), increased number of root branches, increased total root length, efficiency of nodulation and nitrogen fixation, enhanced nitrogen use efficiency, increased water use efficiency as compared to a control plant, efficiency of nutrient assimilation, resistance to biotic and abiotic stress, carbon assimilation, physiology, enhanced disease or pest resistance, or environmental or chemical tolerance, enhanced cold tolerance, nutritional enhancement, enhanced seed protein, enhanced seed starch, enhanced seed oil, plant height, enhanced plant morphology, growth and development, and stay green rating.

In some embodiments, the non-genic plant sequence is a soybean genomic sequence or a corn genomic sequence. In some embodiments, the corn genomic sequence is selected from the group consisting of SEQ ID NOs:123-172, 294, 299-551, 555, and 556. In other embodiments, the soybean genomic sequence is selected from the group consisting of SEQ ID NOs:251-282, 554.

In some embodiments, the target site is located within a small genomic region (e.g., less than 500 bp, less than 1000 bp, less than 2000 bp) of low genetic diversity, where low genetic diversity is defined as having between one, two, three, four, five, six, seven, eight, nine and ten distinguishable haplotypes across all germplasm in an intended heterotic group, an intended maturity group, or an intended heterotic and maturity group.

In some embodiments, the target site comprises at least 75, at least 80, at least 80, at least 100, at least 200, at least 300, at least 400, at least 500, at least 600, at least 700, at least 800, at least 900, or at least 1000 nucleotides. In some embodiments, the target site comprises about 100, about 150, about 200, about 250, about 300, about 350, about 400, about 450, about 500, about 550, about 600, about 650, about 700, about 750, about 800, about 850,about 900, about 950, about 1000, about 1100, about 1200, about 1300, about 1400, about 1500, about 1600, about 1700, about 1800, about 1900, or about 2000 nucleotides.

In one aspect, the haploid window is defined by genetic distance. In some embodiments, the genetic distance is from about 0.1 cM to about 5 cM. In some embodiments, the genetic distance is about 0.1 cM, about 0.2 cM, about 0.3 cM, about 0.4 cM, about 0.5 cM, about 0.6 cM, about 0.7 cM, about 0.8 cM, about 0.9 cM, about 1 cM, about 1.5 CM, about 2cM, about 2.5 CM, about 3 cM, about 3.5 CM, about 4 cM, about 4.5 cM, or about 5 cM.

In some embodiments, the haplotype window is based on physical distance of the haplotype window which is from about 40 base pairs to the full length of the chromosome, with at least 99% sequence similarity across germplasm and contains two or fewer indels of ˜3 kb.

In some embodiments, the target site is further located at a genetic distance of greater than 1 cM, greater than 2 cM, greater than 3 cM, greater than 4 cM, greater than 5 cM, greater than 6 cM, greater than 7 cM, greater than 8 cM, greater than 9 cM, or greater than 10 cM of a haplotype window that is associated with a negative impact on one or more agronomic traits.

In some embodiments, the target site comprises one or more of the criteria selected from the group consisting of: the target site is located greater than 2 kb from a 5′ or a 3′ end of a gene in the plant genome; the target site is located more than 1 kb from a 5′ or a 3′ end of a repeat region in the plant genome, and wherein the repeat region is at least 2 kb in length; the target site is located more than 1 kb from a 5′ or a 3′ end of a repressive chromatin mark in the plant genome; the target site is located more than 200 bases from a small RNA (sRNA) hotspot in the plant genome, and wherein the sRNA hotspot is a sequence from 0.2 to 1 kb in length; the target site is within a region of the plant genome of low a DNA methlyation; the target site is not within a region of the plant genome associated with at least one methylation read containing an MspJi motif or a LpnPI motif; and the target site is within a region of the plant genome that exhibits a total k-mer redundancy score of less than or equal to 30%. In some embodiments, the total k-mer redundancy score is less than or equal to 30%, 29%, 28%, 27%, 26%, 25%, 24%, 23%, 22%, 21%, 20%, 19%, 18%, 17%, 16%, 15%, 14%, 13%, 12%, 11%, or 10%. In some embodiments, the target site is within a 100 bp, 150 bp, 200 bp, 250 bp, 300 bp, 350 bp, 400 bp, 500 bp, 550 bp, 600 bp, 650 bp, 700 bp, 750 bp, 800 bp, 850 bp, 900 bp, 950 bp, or 1,000 bp region of the plant genome that exhibits redundancy score of less than or equal30%, 29%, 28%, 27%, 26%, 25%, 24%, 23%, 22%, 21%, 20%, 19%, 18%, 17%, 16%, 15%, 14%, 13%, 12%, 11%, or 10%.

As used herein, the term “repeat region” refers to a region that is identified by alignment of the host sequence to an annotated second sequence comprising repeat regions wherein the annotation is compiled with genomic repeat identification software.

As used herein, the term “repressive chromatin mark” refers to a statistically significant H3K27me3 (p-value≤5e−3) peak using ChIP-seq peak calling software.

As used herein, the term “small RNA (sRNA) hotspot” refers to a sequence location from 0.2 to 1 Kb in length with statistically significant sRNA abundance (p-value≤5e−3) relative to population average. To identify sRNA hotspots, germplasm-specific sRNA transcripts 21, 22, and 24 nucleotides long, with calculated abundances at least 1 RPM (read per million), are mapped to the genomic sequence to identify regions of high sRNA abundance (Heisel et al., (2008) PLOS ONE 3(8):1-10).

As used herein, the term “DNA methylated region” refers to a locus with a total number of overlapping, but not identical, methylation reads that represent at least 6% of methylation reads identified in the population average of the methylated region.

As used herein, the term “MspJi motif” refers to the consensus genomic sequence CNNR[N]16 (SEQ ID NO:552: cnnrnnnnnn nnnnnnnnnn; where for (2) . . . (3), n is a, c, g, or t; and for (5) . . . (20), n is a, c, g, or t). The term “LpnPI motif” refers to the consensus genomic sequence CSD [N] 16 (SEQ ID NO:553: csdnnnnnnn nnnnnnnnn; where for (4) . . . (19), n is a, c, g, or t). MspJI is a modification-dependent restriction endonuclease that cleaves at a fixed distance away from the modification site. MspJI homologs include, but are not limited to, FspEI, LpnPI, AspBHI, RlaI, and SgrTI. All the enzymes specifically recognize cytosine C5 modification (methylation or hydroxymethylation) in DNA and cleave at a constant distance (N12/N16) away from the modified cytosine. Each MspJI homolog displays its own sequence context preference, favoring different nucleotides flanking the modified cytosine.

As used herein, the term “k-mer redundancy score” is used to calculate a genome redundancy score that quantifies the likelihood of an unique site for site-specific genome modification enzyme cutting, with little off-target effect. The total redundancy score for a selected genomic region is calculated as the percentage of redundant k-mers present in the region. As used herein, “total redundancy score” is calculated as the number of redundant k-mers (having a redundancy score>1) in the region, divided by the total number of k-mers in that region (1000-k for a 1 Kb region), multiplied by 100. In one aspect of the present disclosure, genomic regions with a total redundancy score of 30 or lower (at least 70% of the k-mers in the intergenic region are unique) is accepted for consideration. In some embodiments, genomic regions with a total redundancy score of 35, 30, 25, 20, 15, 10, 5 or lower is accepted for consideration.

In some embodiments, the agronomic trait is selected from one or more of the group consisting of: yield, car relative maturity, car height, car number, increased car size, grain moisture, increased car dry weight per plant, increased number of kernels per ear, increased weight per kernel, increased number of kernels per plant, decreased car void, extended grain fill period, test weight, pod number, number of seed per pod, pod position on the plant, number of internodes, incidence of pod shatter, grain size, decreased days from planting to maturity, increased stalk size, increased number of leaves, increased plant height growth rate in vegetative stage, plant architecture, resistance to lodging, percent seed germination, seedling vigor, juvenile traits, efficiency of germination (including germination in stressed conditions), growth rate (including growth rate in stressed conditions), increased number of root branches, increased total root length, efficiency of nodulation and nitrogen fixation, enhanced nitrogen use efficiency, increased water use efficiency as compared to a control plant, efficiency of nutrient assimilation, resistance to biotic stress, resistance to abiotic stress, carbon assimilation, physiology, enhanced disease or pest resistance, or environmental or chemical tolerance, enhanced cold tolerance, nutritional enhancement, enhanced seed protein, enhanced seed starch, enhanced seed oil, plant height, enhanced plant morphology, growth and development, and stay green rating.

In some embodiments, the donor sequence comprises a gene expression cassette comprising a sequence selected from an insecticidal resistance gene, a herbicide tolerance gene, a nitrogen use efficiency gene, a water use efficiency gene, a nutritional quality gene, a DNA binding gene, a selectable marker gene, and any combination thereof.

In some embodiments, the donor sequence is an exogenous sequence. In some embodiments, the donor sequence comprises an expression cassette.

In some embodiments, the donor sequence comprises a nucleotide sequence that contains at least one functional element, where the functional element is capable of assisting in the insertion, the expression, or the identification of the donor sequence. In some embodiments, the functional element is a promoter, a selectable marker gene, or a targeting sequence.

In some embodiments, the DNA of interest is integrated into the target site via a homologous recombination. In other embodiments, the DNA of interest is integrated into the target site via a non-homologous end joining.

In some embodiments, DNA of interest is integrated into the target site via a site-specific genome modification enzyme. In some embodiments, the site-specific genome modification enzyme is selected from an endonuclease, a recombinase, a transposase, and any combination thereof. In some embodiments, the endonuclease is selected from a meganuclease, a zinc finger nuclease, a transcription activator-like effector nuclease (TALEN), a Cas9 nuclease, a Cpf1 nuclease, a Cas12a nuclease, a Cas12e nuclease, a CasX nuclease, a Cas12d nuclease, a CasY nuclease, a Cas12b nuclease, a C2C1 nuclease, a Cas12c nuclease, a C2C3 nuclease, a C2C4 nuclease, a C2C5 nuclease, a C2C6 nuclease, a C2C7 nuclease, a C2C8 nuclease, a C2C9 nuclease, a C2C10 nuclease, a Cas13a nuclease, a Cas13b nuclease, and a Cas13c nuclease. In some embodiments, the recombinase is a tyrosine recombinase attached to a DNA recognition motif, or a serine recombinase attached to a DNA recognition motif. In some embodiments, the tyrosine recombinase attached to a DNA recognition motif is selected from the group consisting of a Cre recombinase, a Flp recombinase, and a Tnp1 recombinase. In some embodiments, the serine recombinase attached to a DNA recognition motif is selected from the group consisting of a PhiC31 integrase, an R4 integrase, and a TP-901 integrase. In some embodiments, the transposase is a DNA transposase attached to a DNA binding domain.

In some embodiments, a TALEN target site comprises a 5′-TALEN binding site, a spacer sequence, and a 3′TALEN binding site. In some embodiments, the TALEN binding sites within the TALEN target site of corn genomic regions is selected from the SEQ ID NOs presented in Table 1. In some embodiments, the TALEN binding sites within the TALEN target site of soybean genomic region is selected from the SEQ ID NOs presented in Table 2.

The present disclosure also provides a plant, plant cell, or plant part comprising a recombinant sequence as disclosed herein.

In certain embodiments, the plant is selected from: alfalfa, aneth, apple, apricot, artichoke, arugula, asparagus, avocado, banana, barley, beans, beet, blackberry, blueberry, broccoli, brussel sprouts, cabbage, canola, cantaloupe, carrot, cassava, cauliflower, celery, cherry, cilantro, citrus, clementine, coffee, corn, cotton, cucumber, Douglas fir, eggplant, endive, escarole, eucalyptus, fennel, figs, gourd, grape, grapefruit, honey dew, jicama, kiwifruit, lettuce, leeks, lemon, lime, Loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, raspberry, rice, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini plants.

Methods of transforming plants and plant cells are well known by persons of ordinary skill in the art. For instance, specific instructions for transforming plant cells by microprojectile bombardment with particles coated with recombinant DNA are found in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat); U.S. Pat. No. 6,002,070 (rice); U.S. Pat. No. 7,122,722 (cotton); U.S. Pat. No. 6,051,756 (Brassica); U.S. Pat. No. 6,297,056 (Brassica); US Patent Publication 20040123342 (sugarcane) and Agrobacterium-mediated transformation is described in U.S. Pat. No. 5,159,135 (cotton); U.S. Pat. No. 5,824,877 (soybean); U.S. Pat. No. 5,591,616 (corn); U.S. Pat. No. 6,384,301 (soybean); U.S. Pat. No. 5,750,871 (Brassica); U.S. Pat. No. 5,463,174(Brassica); and U.S. Pat. No. 5,188,958 (Brassica), all of which are incorporated herein by reference. Methods for transforming other plants can be found in, for example, Compendium of Transgenic Crop Plants (2009) Blackwell Publishing. Any appropriate method known to those skilled in the art can be used to transform a plant cell with any of the nucleic acid molecules provided herein.

In one aspect, a plant cell provided herein is stably transformed. As used herein, “stably transformed” refers to a transfer of DNA into a genome of a targeted cell that allows the targeted cell to pass the transferred DNA to the next generation. In another aspect, a plant cell provided herein is transiently transformed. As used herein, “transiently transformed” is defined as a transfer of DNA into a cell that is not integrated into a genome of the transformed cell.

In an aspect, a plant cell provided herein is selected from the group consisting of an Acacia cell, an alfalfa cell, an aneth cell, an apple cell, an apricot cell, an artichoke cell, an arugula cell, an asparagus cell, an avocado cell, a banana cell, a barley cell, a bean cell, a beet cell, a blackberry cell, a blueberry cell, a broccoli cell, a Brussels sprout cell, a cabbage cell, a canola cell, a cantaloupe cell, a carrot cell, a cassava cell, a cauliflower cell, a celery cell, a Chinese cabbage cell, a cherry cell, a cilantro cell, a citrus cell, a clementine cell, a coffee cell, a corn cell, a cotton cell, a cucumber cell, a Douglas fir cell, an eggplant cell, an endive cell, an escarole cell, an eucalyptus cell, a fennel cell, a fig cell, a forest tree cell, a gourd cell, a grape cell, a grapefruit cell, a honey dew cell, a jicama cell, kiwifruit cell, a lettuce cell, a leek cell, a lemon cell, a lime cell, a Loblolly pine cell, a mango cell, a maple tree cell, a melon cell, a mushroom cell, a nectarine cell, a nut cell, an oat cell, an okra cell, an onion cell, an orange cell, an ornamental plant cell, a papaya cell, a parsley cell, a pea cell, a peach cell, a peanut cell, a pear cell, a pepper cell, a persimmon cell, a pine cell, a pineapple cell, a plantain cell, a plum cell, a pomegranate cell, a poplar cell, a potato cell, a pumpkin cell, a quince cell, a radiata pine cell, a radicchio cell, a radish cell, a rapeseed cell, a raspberry cell, a rice cell, a rye cell, a sorghum cell, a Southern pine cell, a soybean cell, a spinach cell, a squash cell, a strawberry cell, a sugar beet cell, a sugarcane cell, a sunflower cell, a sweet corn cell, a sweet potato cell, a sweetgum cell, a tangerine cell, a tea cell, a tobacco cell, a tomato cell, a turf cell, a vine cell, watermelon cell, a wheat cell, a yam cell, and a zucchini cell. In another aspect, a plant cell provided herein is selected from the group consisting of a corn cell, a soybean cell, a canola cell, a cotton cell, a wheat cell, and a sugarcane cell.

In another aspect, a plant cell provided herein is selected from the group consisting of a corn immature embryo cell, a corn mature embryo cell, a corn seed cell, a soybean immature embryo cell, a soybean mature embryo cell, a soybean seed cell, a canola immature embryo cell, a canola mature embryo cell, a canola seed cell, a cotton immature embryo cell, a cotton mature embryo cell, a cotton seed cell, a wheat immature embryo cell, a wheat mature embryo cell, a wheat seed cell, a sugarcane immature embryo cell, a sugarcane mature embryo cell, a sugarcane seed cell.

In one aspect, plant cells disclosed herein include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a stomata cell, a trichome cell, a root hair cell, a storage root cell, or a tuber cell. In another aspect, this disclosure provides a protoplast. In another aspect, this disclosure provides a plant callus cell.

In one aspect, the instant disclosure provides a plant, plant cell, or plant part that is transformed by any method provided herein.

To confirm the presence of integrated DNA in a transformed cell or genome a variety of assays can be performed. Such assays include, for example, molecular biological assays (e.g., Southern and northern blotting, PCR); biochemical assays, such as detecting the presence of a protein product (e.g., by immunological means (ELISAs and western blots), or by enzymatic function (e.g., GUS assay); pollen histochemistry; plant part assays, (e.g., leaf or root assays); and, by analyzing the phenotype of the whole regenerated plant.

### Site-Specific Genome Modification Enzymes

As used herein, the term “double-strand break inducing agent” refers to any agent that can induce a double-strand break (DSB) on a DNA molecule. In some embodiments, the double-strand break inducing agent is a site-specific genome modification enzyme.

As used herein, the term “site-specific genome modification enzyme” refers to any enzyme that can modify a nucleotide sequence in a site-specific manner. In the present disclosure, site-specific genome modification enzymes include endonucleases, recombinases, transposases, helicases and any combination thereof.

Several embodiments relate to promoting recombination by providing a site-specific genome modification enzyme. As used herein, the term “site-specific enzyme” refers to any enzyme that can modify a nucleotide sequence in a site-specific manner. In some embodiments, recombination is promoted by providing a single-strand break inducing agent. In some embodiments, recombination is promoted by providing a double-strand break inducing agent. In some embodiments, recombination is promoted by providing a strand separation inducing reagent. In one aspect, the site-specific genome modification enzyme is selected from an endonuclease, a recombinase, a transposase, a helicase or any combination thereof.

In one aspect, the endonuclease is selected from a meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csnl and Csx12), Cas10, Cal2a, Cas12b, Cas12e, Cas12d, Cas13a, Cas13, Cas13c, Csyl, Csy2, Csy3, Csel, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2C1, C2C3, C2C4, C2C5,

C2C6, C2C7, C2C8, c2C9, C2C10, CasX, CasY homologs thereof, or modified versions thereof).

In another aspect, the endonuclease is a dCas9-recombinase fusion protein. As used herein, a “dCas9” refers to a Cas9 endonuclease protein with one or more amino acid mutations that result in a Cas9 protein without endonuclease activity, but retaining RNA-guided site-specific DNA binding. As used herein, a “dCas9-recombinase fusion protein” is a dCas9 with a protein fused to the dCas9 in such a manner that the recombinase is catalytically active on the DNA.

Non-limiting examples of recombinase include a tyrosine recombinase attached to a DNA recognition motif provided herein is selected from the group consisting of a Cre recombinase, a Gin recombinase a Flp recombinase, and a Tnpl recombinase. In an aspect, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

Site-specific genome modification enzymes, such as meganucleases, ZFNs, TALENs, Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof), RNA-guided nucleases (non-limiting examples of RNA-guided nucleases include the CRISPR associated nucleases, such as Cas1, Cas1B, Cas2, Cas3, Cas4, Cas5, Cas6, Cas7, Cas8, Cas9 (also known as Csnl and Csx12), Cas10, Cal2a, Cas12b, Cas12c, Cas12d, Cas13a, Cas13, Cas13c, Csy1, Csy2, Csy3, Csel, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2C1, C2C3, C2C4, C2C5, C2C6, C2C7, C2C8, c2C9, C2C10, CasX, CasY, homologs thereof, or modified versions thereof) and engineered RNA-guided nucleases (RGNs), induce a genome modification such as a double-stranded DNA break (DSB) or single-strand DNA break at the target site of a genomic sequence that is then repaired by the natural processes of homologous recombination (HR) or non-homologous end-joining (NHEJ). Sequence modifications then occur at the cleaved sites, which can include deletions or insertions that result in gene disruption in the case of NHEJ, or integration of exogenous sequences by homologous recombination.

In one aspect of the present disclosure, site-specific genome modification enzymes are selected to induce a genome modification in one, a few, or many individual target sequences of the plant genomic sequences provided herein. After exposure to the site-specific genome modification enzyme, the resulting recombinant nucleic acid can be identified in various ways including sequencing, PCR amplification, Southern analysis, or other molecular methods used to detect recombinant nucleic acid sequence. Site-specific genome modification enzymes may be expressed in plants such that one or more genome modifications occur within a genomic locus, and resulting progeny screened for molecular changes.

Any of the DNA of interest provided herein can be integrated into a target site of a plant genomic sequence by introducing the DNA of interest and the provided site-specific genome modification enzymes. Any method provided herein can utilize any site-specific genome modification enzyme provided herein.

### ZFNs

Zinc finger nucleases (ZFNs) are synthetic proteins characterized by an engineered zinc finger DNA-binding domain fused to the cleavage domain of the FokI restriction endonuclease. ZFNs can be designed to cleave almost any long stretch of double-stranded DNA for modification of the zinc finger DNA-binding domain. ZFNs form dimers from monomers composed of a non-specific DNA cleavage domain of FokI endonuclease fused to a zinc finger array engineered to bind a target DNA sequence.

The DNA-binding domain of a ZFN is typically composed of 3-4 zinc-finger arrays. The amino acids at positions −1, +2, +3, and +6 relative to the start of the zinc finger ∞-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art.

The FokI nuclease domain requires dimerization to cleave DNA and therefore two ZFNs with their C-terminal regions are needed to bind opposite DNA strands of the cleavage site (separated by 5-7 bp). The ZFN monomer can cute the target site if the two-ZF-binding sites are palindromic. The term ZFN, as used herein, is broad and includes a monomeric

ZFN that can cleave double stranded DNA without assistance from another ZFN. The term ZFN is also used to refer to one or both members of a pair of ZFNs that are engineered to work together to cleave DNA at the same site.

Because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

### TALENs

Transcription activator-like effectors (TALEs) can be engineered to bind practically any DNA sequence. TALE proteins are DNA-binding domains derived from various plant bacterial pathogens of the genus Xanthomonas. The X pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of cach monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs. The transcription activator-like effector (TALE) DNA binding domain can be fused to a functional domain, such as a recombinase, a nuclease, a transposase or a helicase, thus conferring sequence specificity to the functional domain.

Transcription activator-like effector nucleases (TALENs) are artificial restriction enzymes generated by fusing the transcription activator-like effector (TALE) DNA binding domain to a nuclease domain. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site. In some embodiments, the nuclease is selected from a group consisting of PvuII, MutH, TevI, FokI, AlwI, MlyI, SbfI, SdaI, StsI, CleDORF, Clo051, and Pept071. When FokI is fused to a TALE domain each member of the TALEN pair binds to the DNA sites flanking a target site, the FokI monomers dimerize and cause a DSB at the target site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain, and the number of bases between the two individual TALEN binding sites are parameters for achieving high levels of activity. PvuII, MutH, and TevI cleavage domains are useful alternatives to FokI and FokI variants for use with TALEs. Pvull functions as a highly specific cleavage domain when coupled to a TALE (see Yank et al. 2013. PLOS One. 8: e82539). MutH is capable of introducing strand-specific nicks in DNA (see Gabsalilow et al. 2013. Nucleic Acids Research. 41: c83). TevI introduces double-stranded breaks in DNA at targeted sites (see Beurdeley et al., 2013. Nature Communications. 4:1762).

The relationship between amino acid sequence and DNA recognition of the TALE binding domain allows for designable proteins. Software programs such as DNA Works can be used to design TALE constructs. Other methods of designing TALE constructs are known to those of skill in the art. Doyle et al. (2012) TAL Effector-Nucleotide Targeter (TALE-NT) 2.0: tools for TAL effector design and target prediction. Nucleic Acids Res. 40(W1):W117-W122; Cermak (2011). Efficient design and assembly of custom TALEN and other TAL effector-based constructs for DNA targeting. Nucleic Acids Res. 39(12):e82.

### Meganucleases

Meganucleases, which are commonly identified in microbes, are unique enzymes with high activity and long recognition sequences (>14 bp) resulting in site-specific digestion of target DNA. Engineered versions of naturally occurring meganucleases typically have extended DNA recognition sequences (for example, 14-40 bp).

The engineering of meganucleases is more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

### Argonaute

The Argonaute protein family is a DNA-guided endonuclease. The Argonaute isolated from Natronobacterium gregoryi has been reported to be suitable for DNA-guided genome editing in human cells (Gao, et al. DNA-guided genome editing using the Natronobacterium gregoryi Argonaute. Nature Biotechnology 34:768-773 (2016). Argonaute endonucleases from other species have been identified, (non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof). A sequence encoding a DNA guide is associated with each of these unique Argonaute endonucleases.

### CRISPR

The CRISPR (clustered regularly interspaced short palindromic repeats)/Cas (CRISPR-associated) system is an alternative to synthetic proteins whose DNA-binding domains enable them to modify genomic DNA at specific sequences (e.g., ZFN and TALEN). Specificity of the CRISPR/Cas system is based on an RNA-guide that use complementary base pairing to recognize target DNA sequences. In some embodiments, the site-specific genome modification enzyme is a CRISPR/Cas system. In an aspect, a site-specific genome modification enzyme provided herein can comprise any RNA-guided Cas nuclease (non-limiting examples of RNA-guided nucleases include Cas1, Cas1B, Cas2, Cas3, Cas4, Cas5, Cas6, Cas7, Cas8, Cas9 (also known as Csnl and Csx12), Cas10, Cal2a, Cas12b, Cas12c, Cas12d, Cas13a, Cas13, Cas13c, Csy1, Csy2, Csy3, Csel, Csc2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx 17, Csx 14, Csx 10, Csx 16, CsaX, Csx3, Csx1, Csx 15, Csf1, Csf2, Csf3, Csf4, Cpf1, C2C1, C2C3, C2C4, C2C5, C2C6, C2C7, C2C8, c2C9, C2C10, CasX, CasY, homologs thereof, or modified versions thereof); and, optionally, the tracr and/or guide RNA necessary for targeting the respective nucleases.

CRISPR/Cas systems are part of the adaptive immune system of bacteria and archaca, protecting them against invading nucleic acids such as viruses by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA. A prerequisite for cleavage is the presence of a conserved protospacer-adjacent motif (PAM) downstream of the target DNA, which usually has the sequence 5′-NGG-3′ but less frequently 5′-NAG-3′. Specificity is provided by the so-called “seed sequence” approximately 12 bases upstream of the PAM, which must match between the RNA and target DNA. Cpf1 acts in a similar manner to Cas9, but Cpf1 does not require a separate tracrRNA.

The following Examples are presented for the purposes of illustration and should not be construed as limitations.

### EXAMPLES

### Example 1: Target Site Selection

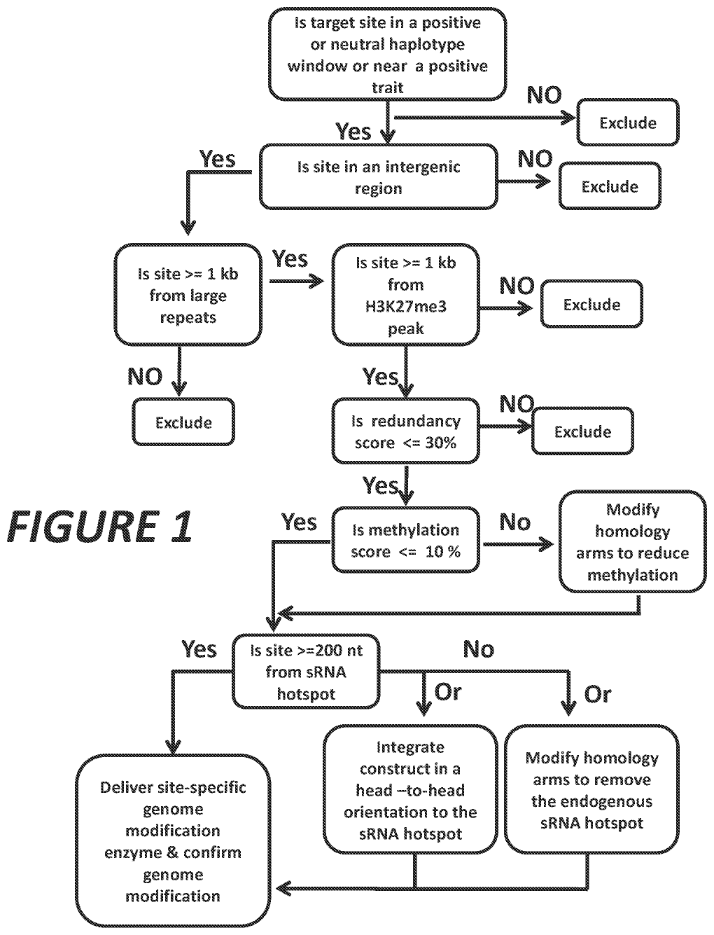

A flowchart for selecting target sites for site-specific genome modification (including, integration of a DNA of interest into a genomic sequence) is shown in FIG. 1. This flowchart illustrates steps that include bioinformatic analysis of a host genome and the application of specific selection criteria to identify target sites for site-specific genome modification. The analysis includes one or more of the following site-specific selection criteria: 1) selection of an initial haplotype window that has a neutral or positive association with an agronomic trait, 2) the target site is within an intergenic region, 3) the target site is greater than or equal to 1 kb away from a long repeat region, 4) the target site is greater than or equal to 1 kb away from a repressive chromatin mark (e.g., H3K27me3 peak), 5) the target site is within a region with a low redundancy score (less-than or equal to 30%), 6) the target site is within a region with a low DNA methylation score (less-than or equal to 10% of genome wide population average), 7) the target site is greater-than or equal to 200 bp away from a small RNA (sRNA) hotspot, 8) selecting areas targetable by site-specific genome modification enzymes.

For case of presentation, the target site selection process is presented as the flowchart in FIG. 1. The steps in which the target site selection criteria are completed may be in any order. For example, the step of determining if a target site is within a region with a low redundancy score (less-than or equal to 30%) may be completed prior the step of determining whether the target site is within an intergenic region. In some instances, not all the criteria will be used to select a target site.

The first step shown in FIG. 1 is the selection of a haplotype window that has a neutral or positive association with an agronomic trait. This haplotype window would be located within a region of low genetic diversity (defined as, ten or fewer haplotypes), and at least 1 cM away from a haplotype window associated with yield drag (or drag of another undesired agronomic trait). The specific target site is selected from a sequence within the low diversity haplotype window. Low genetic diversity is defined as having between one and ten distinguishable haplotypes across all germplasm in the intended heterotic group, the intended maturity group, or the intended heterotic and maturity group, such as disclosed in US Patent Pub. No. 2013/0276173, which is incorporated here in its entirety. Inserting new donor nucleotides near positive haplotype windows within genetic distance of 0.1 cM to 5 cM minimizes the likelihood that trait associations will be disrupted by genetic recombination, and allows trait stacking of favorable genes. Short genetic distance would allow negative traits to be bred out, but only after conducting prolonged and expensive crossing experiments.

Another step in the target site selection process is to select a specific target site that is within an intergenic region. Selection of an intergenic region is done to avoid disruption of endogenous genes. Genomic regions immediately upstream (5′) or downstream (3′) of genes are avoided as sites for genome modification, as these regions may contain regulatory sequences required for proper gene function. Although genes, and regions located less than 2 Kb from either the 5′ or the 3′ end of these genes are avoided as target sites for genome modification, the sequence is included in further analysis steps because the genomic regions could function as a homology region for targeted integration of a DNA of interest by homologous recombination. Bioinformatic analysis is done using publicly available annotation of particular genomes to identify genic regions. Specific target sites are selected that are greater than or equal to 2 Kb from either the 5′- or 3′-end of known genes and within the selected haplotype window. The intergenic regions remaining after discarding coding regions in the haplotype window form the pool of potential sites for the next phase of target site selection.

A further step in the target site selection process is to select a region that is not in or adjacent to genomic repeat regions. Highly repetitive DNA is frequently found in heterochromatic genomic regions. Due to the repeat structure, site-specific genome modification may be inefficient and/or result in reduced agronomic benefit. For example, with integration of a transgenic expression cassette, the repeat region may result in reduced transcription of the transgene cassette resulting in reduced expression of the transgene. For the analysis, the sequence of the genomic regions within selected low diversity haplotype windows are evaluated bioinformatically to identify specific nucleotide coordinates of repeat regions, and then further analyzed by visualization in using Genome Browser tools (Kent et al. (2002) Genome Res. 12(6):996-1006). Genomic regions located in repeat regions greater than 2 Kb long plus a 1 Kb buffer zone on either end of the repeat region (4 kb total) are excluded for specific target site selection. Repeat regions less than 2 Kb long are included in the pool of potential target sites.

A further step in the target site selection process is to select a region that is lacking in repressive chromatin marks. Histones are the primary protein components of chromatin, and H3K27me3 is a well-known histone H3 modification that is associated with facultatively repressed genes. H3K27me3 levels in corn were identified using the ChIP-seq method followed by Illumina sequencing (Deng, J. et al. (2009) Nat. Biotechnol. 27, 353-360). The sequence of the genomic regions within the selected low diversity haplotype windows are evaluated with MACS software (Zhang et al. (2008) Genome Biol. 9 (9): R137) to identify sequence predicted to have chromatin peaks based on the ChIP-seq analysis. Specific target sites were selected which were greater than or equal to 1 kb away from these repressive chromatin marks.

Another step in the target site selection process is to select a target site that is within a region with a low redundancy score (less-than or equal to 30%). A redundancy score is a mathematical measure of the likelihood that the sequence is unique in the genome. The redundancy score is calculated by a using a binned k-mer approach where a k-mer window is selected and a scanning window is used to shift the k-mer window 1-nucleotide in the 3′ direction along the entire host genome. The k-mer redundancy count is calculated by summing the number of times there is a perfect nucleotide match (100% sequence identify) for each k-mer sequence in the host genome. A unique k-mer has a redundancy count equal to 1, as this nucleotide sequence occurs exactly once in the reference genome. A redundant k-mer has a redundancy count of greater than 1, as this nucleotide sequence occurs more than once in the reference genome.

The total redundancy score for a selected genomic region is calculated as the percent of redundant k-mers present in a specific genomic region. For example, a total redundancy score for genomic region at least 1000 nucleotides long is selected for analysis, and the total number of unique k-mers (k-mer redundancy count of 1) vs. redundant k-mers (k-mer redundancy count greater than 1) are calculated. The total number of k-mers within the 1000 nucleotide region is equal to (1000-k), where k is the number of nucleotides in each k-mer. The total redundancy score is calculated as the number of redundant k-mers in a region, divided by the total number of k-mers in that region, multiplied by 100. A total redundancy score of 30% indicates that at least 70% of the k-mers in the genomic region are unique.

Another step in the target site selection process is to select sites within low DNA methylation regions. DNA methylation is a common epigenetic mechanism to reduce gene expression. DNA methylation has been reported to interfere with TALEN activity (Bultmann S., et al. (2012) Targeted transcriptional activation of silent oct4 pluripotency gene by combining designer TALEs and inhibition of epigenetic modifiers. Nucleic Acids Res. 40, 5368-5377). DNA methylation regions were identified by digesting corn genomic DNA with a cocktail of DNA-methylation sensitive enzymes per supplier protocols (New England Biolabs, Ipswich, MA). The DNA fragments (20-40 nucleotides long) were extracted, and a DNA library was prepared using the ThruPLEX-FD® DNA-seq kit (Rubicon Genomics, Ann Arbor, MI). Next, libraries were sequenced using the TruSeq® DNA Methylation kit (Illumina Inc., San Diego, CA), and DNA methylation reads were mapped to their corresponding loci on the corn genome. Genomic loci associated with a cluster of overlapping but non-identical reads that represent 10% of genome wide population average are classified as DNA methylated regions and were excluded from target site selection. In other examples, a cluster of at least four overlapping but non-identical reads were classified as a DNA methylated region and were excluded from target site selection.

Another step in the target site selection process is to select target sites≥200 bp away from a small RNA (sRNA) hotspot, where a sRNA hotspot is a region with high sRNA abundance. A sRNA hotspot may function as sRNA binding sites if this region of the genome is included in pre-mRNA transcripts generated during transcription of genes (mRNA transcripts from either endogenous genes or from transgene cassettes) in the vicinity of sRNA hotspots. To identify sRNA hotspots, germplasm-specific sRNA transcripts 21, 22, and 24nucleotides long, with calculated abundances at least 1 RPM (read per million), are mapped to the genomic sequence to identify regions of high sRNA abundance (Heisel et al., (2008) PLOS ONE 3(8) 1-10). For the purpose of genome modification, including site-specific integration of a DNA of interest, the target site selected is positioned≥200 bp away from a small RNA (SRNA) hotspot.

In some instances, a target site is selected that is in a region (≤200 nucleotides) of an sRNA hotspot. If the target site is selected for integration of a transgene cassette, the orientation of integration of the transgene cassette can be designed such that the sRNA hotspot is in a ‘head-to-head’ orientation with the transgene cassette. This ‘head-to-head’ orientation is where the direction of transcription of the transgene cassette is in the opposite orientation of the direction of transcription of the sRNA hotspot within the genome. This head-to-head orientation will reduce the chance of incorporation of sRNA binding sites during transcription of mRNA from the transgene cassette.

When the target site is in a region (≤200 nucleotides) of an sRNA hotspot and the target site is selected for homology-dependent integration of a transgene cassette, then design of one or both homology arms of the transgene cassette is done to remove the sRNA hotspot during integration at the target site. Specifically, the homology arms of the transgene cassette are designed to have homology to a genomic within the sRNA hotspot, or flanking on the distal 5′-end (for a 5′ homology arm) or the distal 3′-end (for a 3′ homology arm) of the sRNA hotspot, and during the HR-dependent integration of the transgene cassette the process of homologous recombination effectively truncates and/or deletes the sRNA hotspot from the final transgenic genomic locus.

### Example 2: Site-Specific Genome Modification

The specific target site identified using the target site selection process detailed in Example 1 is used to inform the process of site-specific genome modification. For example, a site-specific genome modification enzyme delivery system is engineered and delivered to the plant cell.

In one example, a meganuclease is engineered to bind at the specific target site selected for genome modification. The sequence encoding the meganuclease is cloned into a plant expression vector, and delivered to the plant cell. If the genome modification is designed to induce a double-strand break (DSB) with non-homologous end joining (NHEJ) repair for introduction of insertions and deletions (indels), then just the engineered meganuclease is delivered to the plant cell. If a DNA of interest is to be incorporated at the target site, then the engineered meganuclease and the DNA of interest are co-delivered to the plant cell. The DNA of interest may integrate by NHEJ or by homology-dependent repair (HR). In the latter case, the DNA of interest will have at least one homology arm.

In another example, a Zinc Finger Nuclease (ZFN) is used to introduce the site-specific genome modification. In this case, the pair of ZFN molecules are designed and cloned into a plant expression vector and delivered to the plant cell. If the genome modification is designed to induce a double-strand break (DSB) with non-homologous end joining (NHEJ) repair for introduction of insertions and deletions (indels), then just the engineered ZFN is delivered to the plant cell. If a DNA of interest is to be incorporated at the target site, then the engineered ZFN and the DNA of interest are co-delivered to the plant cell. The DNA of interest may integrate by NHEJ or by homology-dependent repair (HR). In the latter case, the DNA of interest will have at least one homology arm.

In another example, a TAL-effector nuclease (TALEN) is used to introduce the site-specific genome modification. In this case, the pair of TALEN molecules are designed and cloned into a plant expression vector and delivered to the plant cell. A variety of tools known to one skilled in the art are available to design a TALEN for optimal activity for a selected target site. One example of a tool for TALEN design is described by Lin et al., (2014) Nucleic Acids Res. 2014 April; 42(6); and U.S. Patent Application Publication 20150132821. If the genome modification is designed to induce a double-strand break (DSB) with non-homologous end joining (NHEJ) repair for introduction of insertions and deletions (indels), then just the engineered TALEN is delivered to the plant cell. If a DNA of interest is to be incorporated at the target site, then the engineered TALEN and the DNA of interest are co-delivered to the plant cell. The DNA of interest may integrate by NHEJ or by homology-dependent repair (HR). In the latter case, the DNA of interest will have at least one homology arm.

In another example, an Argonaute is used to introduce the site-specific genome modification. In this case, the Argonaute molecule and a DNA guide molecule are designed and cloned into a plant expression vector and delivered to the plant cell. If the genome modification is designed to induce a double-strand break (DSB) with non-homologous end joining (NHEJ) repair for introduction of insertions and deletions (indels), then just the engineered Argonaute and DNA guide molecule are delivered to the plant cell. If a DNA of interest is to be incorporated at the target site, then the engineered Argonaute, DNA guide molecule, and the DNA of interest are co-delivered to the plant cell. The DNA of interest may integrate by NHEJ or by homology-dependent repair (HR). In the latter case, the DNA of interest will have at least one homology arm.

In another example, a CRISPR system is used to introduce the site-specific genome modification. In this case, the CRISPR associated nuclease and at least one RNA guide molecule are designed and cloned into a plant expression vector and delivered to the plant cell. If the genome modification is designed to induce a double-strand break (DSB) with non-homologous end joining (NHEJ) repair for introduction of insertions and deletions (indels), then just the engineered CRISPR nuclease and at least one RNA guide molecule are delivered to the plant cell. The RNA guide molecule may be a single guide RNA (sgRNA) or the RNA guide molecule may have both a tracer-RNA and guide-RNA component. If a DNA of interest is to be incorporated at the target site, then the engineered CRISPR nuclease, at least one RNA guide molecule, and the DNA of interest are co-delivered to the plant cell. The DNA of interest may integrate by NHEJ or by homology-dependent repair (HR). In the latter case, the DNA of interest will have at least one homology arm. An alternative to delivery of the engineered CRISPR nuclease as a DNA expression construct is the delivery of a Ribonucleo-protein (RNP) complex of the CRISPR associated nuclease protein in complex with the guide RNA.

Following delivery of the genome modification system to a plant cell, the cells or plants regenerated from the cells are sampled to confirm the presence of the intended site-specific genome modification. Methods of detecting the genome modification are known to one skilled in the art, and include: PCR, TaqMan® PCR, droplet digital PCR (ddPCR™, Bio-Rad Laboratories, Hercules, CA), sequencing, Sanger sequencing, ABI 3730 DNA fragment analysis (Applied Biosystems, Grand Island, NY), Southern analysis, Northern analysis, phenotypic analysis, or any other technique known to one in the art to detect genome modification.

Example 3: TALEN Target Site Selection in Corn

The genomic sequences for three separate corn germplasm (B73, 01DKD2, and LH244) were analyzed using the criteria detailed in Example 1 to identify specific target sites for genome modification by TALENs. From the analysis of the chromosome I genomic locus the B73 germplasm (chosen as being in a haplotype window associated with positive agronomic trait of insect resistance), 17 genomic regions containing TALEN targeting sites were identified, represented by SEQ ID NO:140 through SEQ ID NO:156. Using these 17 regions from the B73 germplasm analysis, 16 corresponding genomic regions containing TALEN targeting sites were identified in 01DKD2 germplasm, represented by SEQ ID NO:157 through SEQ ID NO:172. Using the 17 regions from the B73 germplasm analysis, 17 corresponding genomic regions in LH244 germplasm were identified, represented by SEQ ID NO:123 through SEQ ID NO:139. For each of the 17 genomic regions in the LH244 germplasm, there were from one to six separate TALEN target sites per genomic region, with a total of 61 separate TALEN targeting sites selected for testing TALEN activity (see Table 1).

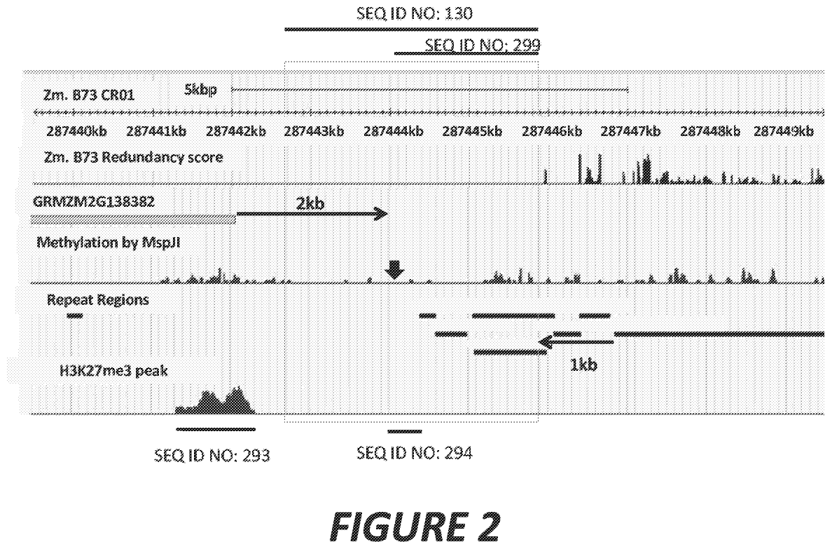

As one representative example of the target site selection process, a genomic region on corn chromosome 1, represented by SEQ ID NO:130, was chosen initially as being within a haplotype window associated with a transgene insertion event with a positive agronomic trait. This haplotype window was approximately 36 Mb in length and was identified essentially as described in US Patent Pub. No. 20130276173. The relative position of SEQ ID NO: 130 within a 10 kb region of the haplotype window is illustrated in FIG. 2.

As detailed in Example 1, the genomic sequence within the haplotype window was analyzed for genic and intergenic coordinates. From this analysis, an exon for a gene identified as GRMZM2G138382 was identified within the 10 kb window selected for analysis to identify a TALEN target site. Based on this analysis, and applying the criteria to include/exclude genic regions as detailed in Example 1, a genomic sequence of approximately 5 kb in length, as illustrated in FIG. 2, between Zm.B73 CR01 coordinates 287442 kb to 287447 kb, was selected for further analysis to identify a TALEN target site using additional selection criteria as detailed below.

As detailed in Example 1, the selected 5 kb genomic sequence was analyzed for regions of repetitive sequence. The reference corn genome for LH244 was analyzed in Genome Browser (Kent et al. (2002) Genome Res. 12(6):996-1006) and known repeat regions occurring within the selected 5 kb sequence were mapped. The analysis window from Genome Browser was inspected to identify repeat regions greater than 2 kb in length. One large repeat occurred within the preselected 10 kb region and this repeat plus 1 kb upstream, illustrated in FIG. 2, Zm.B73 CR01 between coordinates 287445.9 kb to 287229 kb, were excluded from further analysis. Combining the analysis of the haplotype window, the genic/intergenic regions, and the repetitive sequence, a genomic sequence of 3.5 kb (SEQ ID NO:130) was selected as a region to identify a TALEN target site, with the 1.6 kb sequence (SEQ ID NO:299) selected as the optimal region, thus avoiding the endogenous gene plus 2 kb buffer (Zm.B73 CR01 coordinates 287444 kb to 287445.9 kb), as illustrated in FIG. 2. This sequence was selected for further analysis to identify a TALEN target site using additional selection criteria as detailed below.

As detailed in Example 1, an analysis was done for the presence of repressive chromatin marks, assessed by H3K27me3 peaks. The nearest H3K27me3 peak occurred at the sequence identified by SEQ ID NO:293 (FIG. 2) positioned about 2 kb upstream of the genomic region selected to identify a TALEN target site (SEQ ID NO:299). Therefore, further analysis was done for the region represented by SEQ ID NO:299 to identify a TALEN target site using additional selection criteria as detailed below.

The genomic sequence (SEQ ID NO:299) was analyzed to identify sRNA binding sites as detailed in Example 1. Within the genomic sequence of the selected site (SEQ ID NO:299), two 24 nt sRNA hotspots were identified. One sRNA hotspot occurred approximately 160 bp upstream of the SEQ ID NO:299, and one sRNA hotspot occurred approximately 1400 bp downstream of the SEQ ID NO:299. Due to the proximity (160 bp) of the upstream sRNA hotspot, a transgene cassette is designed to integrate by homologous recombination into the TALEN target site in a head-to-head orientation relative to this sRNA hotspot. This orientation of the transgene cassette would reduce or eliminate run-on by Pol-II polymerase from extending transcription of the transgene mRNA transcript into the sRNA hotspot. Therefore, sRNA binding sites are not transcribed into the nacent transgene mRNA transcript, thus reducing the potential for sRNA induced transgene silencing.

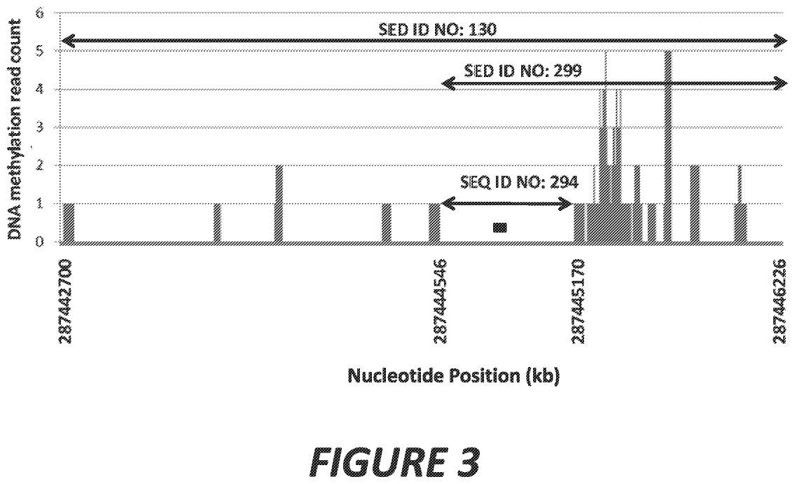

As detailed in Example 1, the DNA methylation status within the genomic region was analyzed. Genome-wide DNA methylation profiling was performed by extracting DNA fragments (20-40 nt long) from corn tissue, and preparing a DNA library using the ThruPLEX-FD® DNA-seq kit (Rubicon Genomics, Ann Arbor, MI). The DNA libraries were sequenced using the TruSeq® DNA Methylation kit (Illumina Inc., San Diego, CA), and DNA reads were mapped to the sequences represented as SEQ ID NO:130, and SEQ ID NO:299(FIG. 3). Because DNA methylation interferes with TALEN activity, sequence associated with a cluster of at least four overlapping but not identical reads were classified as a DNA methylation region, and were excluded as a TALEN target site. The DNA methylation profile for SEQ ID NO:130 and SEQ ID NO:299 was highly heterogeneous, with DNA methylation read counts varying from 0-5 MspJI/LPnPI read counts across the genomic region, as illustrated in FIG. 3. Due to the relatively high MspJI/LPnPI read counts overlapping SEQ ID NO:299, a region of 530 bp and represented by SEQ ID NO:294 was selected as the genomic region for TALEN induced genome modification.

As detailed in Example 1, the region within the selected haplotype window was analyzed for sequence redundancy, with total redundancy scores across the genomic region determined. To calculate the redundancy scores, the haplotype region was binned using an 18 nucleotide k-mer window, and for each k-mer the redundancy score was calculated. The total redundancy score for the target region was then calculated from the individual k-mer redundancy scores, as described in Example 1. The total redundancy score for SEQ ID NO:130 was 28, marginally below the preferred cut-off value of 30. Upon visual inspection of the entire length of the 530 bp region represented by SEQ ID NO:294, this region had a total redundancy score that was≤8, a value much lower than the cut-off value of 30. The position of the genomic sequences corresponding to SEQ ID NO:130, SEQ ID NO:299, and SEQ ID NO:294 relative to the redundancy score is illustrated in FIG. 2, of the genomic regions.

The final step of the TALEN site selection process was to repeat the redundancy score analysis to identify sequence of at least 200 bp, and that had a total redundancy score of less than 10. This step was added to ensure high TALEN nuclease specificity at the selected target site.

For the entire genomic sequence of corn LH244 germplasm in the haplotype window on chromosome 1 described above, the process to select multiple TALEN target sites was completed largely as described above. This TALEN site selection process identified 61 separate TALEN target sites, represented in 17 genomic sequences (SEQ ID NO:123-139). For each of the 17 genomic sequences, there were from one to six separate specific TALEN targeting sites. A TALEN target site included a 5′-TALEN binding site, a spacer sequence, and a 3′TALEN binding site. Each of the TALEN binding sites were 15 to 24 bp long, and the spacer sequence was 18 to 25 bp long. Within the spacer sequence is the site of the DSB induced by the TALEN, and the site of incorporation of indels or incorporation of a DNA of interest by either NHRJ or HR. The SEQ ID NOs for the 5′-TALEN binding site and 3′-TALEN binding site corresponding to each of the 61 TALEN target sites are represented in Table 1.

TABLE 1
TALEN activity measured by DNA integration at
individual LH244 TALEN target sites.

| Genomic | 5′ TALEN | 3′ TALEN | TALEN activity |
|---|---|---|---|
| region | binding site | binding site | for DNA integration |
| SEQ ID NO: | SEQ ID NO: | SEQ ID NO: | (active/not active) |
| 123 | 5 | 64 | active |
| 123 | 6 | 65 | active |
| 123 | 7 | 66 | active |
| 123 | 8 | 67 | active |
| 124 | 9 | 68 | not active |
| 124 | 10 | 69 | not active |
| 124 | 11 | 70 | active |
| 124 | 12 | 71 | active |
| 125 | 13 | 72 | active |
| 125 | 14 | 73 | active |
| 125 | 15 | 74 | active |
| 126 | 16 | 75 | active |
| 126 | 17 | 76 | active |
| 126 | 18 | 77 | active |
| 126 | 19 | 78 | active |
| 127 | 20 | 79 | not active |
| 127 | 21 | 80 | active |
| 127 | 22 | 81 | active |
| 127 | 23 | 82 | not active |
| 128 | 24 | 83 | active |
| 128 | 25 | 84 | active |
| 128 | 26 | 85 | active |
| 128 | 27 | 86 | active |
| 129 | 28 | 87 | active |
| 129 | 29 | 88 | not active |
| 129 | 30 | 89 | not active |
| 129 | 31 | 90 | active |
| 130 | 32 | 91 | active |
| 130; 299 | 33 | 92 | active |
| 130; 299 | 34 | 93 | active |
| 130; 299; 294 | 35 | 94 | active |
| 131 | 295 | 296 | not active |
| 131 | 297 | 298 | not active |
| 131 | 36 | 95 | active |
| 131 | 37 | 96 | active |
| 131 | 38 | 97 | not active |
| 131 | 39 | 98 | active |
| 132 | 40 | 99 | not active |
| 132 | 41 | 100 | active |
| 132 | 42 | 101 | active |
| 132 | 43 | 102 | active |
| 133 | 44 | 103 | active |
| 133 | 45 | 104 | not active |
| 133 | 46 | 105 | not active |
| 134 | 47 | 106 | not active |
| 134 | 48 | 107 | not active |
| 134 | 49 | 108 | active |
| 135 | 50 | 109 | active |
| 136 | 51 | 110 | active |
| 136 | 52 | 111 | active |
| 136 | 53 | 112 | not active |
| 136 | 54 | 113 | not active |
| 137 | 55 | 114 | active |
| 137 | 56 | 115 | active |
| 137 | 57 | 116 | active |
| 138 | 58 | 117 | active |
| 138 | 59 | 118 | active |
| 138 | 60 | 119 | not active |
| 138 | 61 | 120 | not active |
| 139 | 62 | 121 | not active |
| 139 | 63 | 122 | active |

To assess TALEN activity at each of these TALEN target sites, a TALEN was engineered to bind at each of the 5′-and 3′-TALEN binding sites. For example, within the genomic region corresponding to SEQ ID NO:130, four TALEN target sites were tested having TALEN binding sites: (1) SEQ ID NO:32 and SEQ ID NO:91; (2) SEQ ID NO:33 and SEQ ID NO: 92; (3) SEQ ID NO:34 and SEQ ID NO:93; and (4) SEQ ID NO:35 and SEQ ID NO:94. Within the genomic region corresponding to SEQ ID NO:299, three TALEN target sites were tested having TALEN binding sites: (1) SEQ ID NO:33 and SEQ ID NO:92; (2) SEQ ID NO:34 and SEQ ID NO:94; and (3) SEQ ID NO:35 and SEQ ID NO:94. Within the genomic region corresponding to SEQ ID NO:294, a single TALEN target site was tested having TALEN binding sites SEQ ID NO:35 and SEQ ID NO:94. The assay used to evaluate TALEN activity was integration of a blunt-end, double-stranded DNA (dsDNA) fragment into the DSB created by the TALEN pair at the specific target sites. Individual expression vectors were generated to contain an expression cassette for each TALEN of the TALEN pair to be evaluated. Two expression vectors (one each for the 5′-and 3′-TALEN binding site) were introduced into isolated corn leaf protoplasts essentially as described in patent application publication WO2015131101, with minor modifications. Briefly, complementary ssDNA oligonucleotides (SEQ ID NO:1 and SEQ ID NO:2) were pre-annealed to form a blunt-end, double-stranded DNA (dsDNA) fragment. Transformations of isolated corn leaf protoplasts were performed using standard PEG-protocol, with 50 pmoles of the dsDNA fragment, and two expression vectors (0.1 pmole each), one for each TALEN of the TALEN pair. Protoplast samples transformed in the presence of the dsDNA fragment alone, or with TALEN plasmids lacking dsDNA fragment, were used as negative controls. The experimental groups contained both TALEN pairs for the specific TALEN target site and the dsDNA fragment. The corn protoplasts were harvested 48 hour after transformation, and the genomic DNA was assayed for integration of the dsDNA fragment. Integration of the dsDNA fragment into the genomic DNA was detected using droplet digital PCR (ddPCR) (Bio-Rad Laboratories, Hercules, CA), or by standard PCR and agarose gel electrophoresis to assess PCR amplicons. The dsDNA fragment may have integrated in either a 5′ or 3′ orientation with respect to the 5′-and 3′-ends of the DSB. Therefore, at least two PCR primer sets were run for each TALEN target site where the primer sets contained a primer specific to the dsDNA fragment (SEQ ID NO:3), and a primer specific to either the 5′ side or the 3′ side of the DSB. For the ddPCR, a TaqMan® probe (SEQ ID NO: 4) was included in the PCR reaction mixture. Transformation efficiency of protoplasts was calculated using a control plasmid expressing green fluorescent protein using the method described in patent application publication WO2015131101. TALEN pairs that showed statistically significant integration of targeted dsDNA fragments were identified as active (scc Table 1).

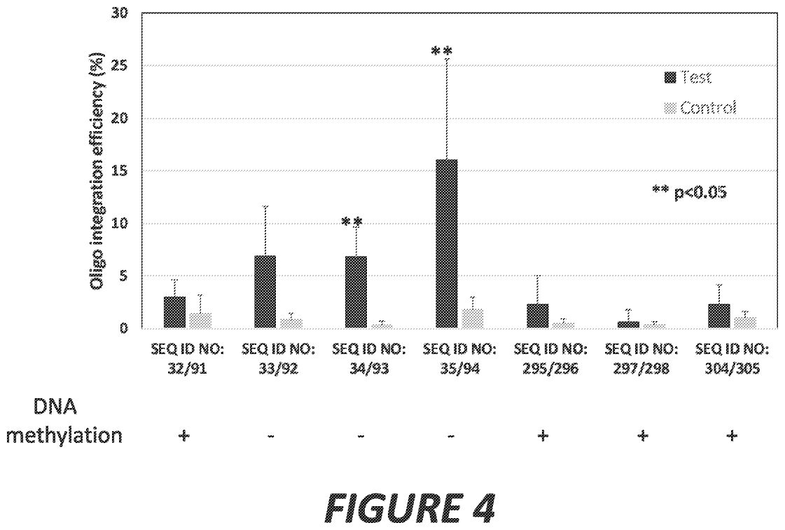

Selection of TALEN target sites that did not overlap DNA methylation regions significantly improved integration of dsDNA fragments into the corn protoplast genome. A comparison of relative DNA methylation region at four separate TALEN target sites and the corresponding percent integration for each of the four TALEN target sites determined in the protoplast assay is shown in FIG. 4. For this comparison, two of the four TALEN target sites, within genomic region SEQ ID NO:130, contain the TALEN binding pairs: SEQ ID NO:34and SEQ ID NO:93; and SEQ ID NO:35 and SEQ ID NO:94; and two of the four TALEN target sites, within genomic region SEQ ID NO:131, contain the TALEN binding pairs: SEQ ID NO:295 and SEQ ID NO:296; and SEQ ID NO:297 and SEQ ID NO:298. The percent integration of the dsDNA fragment into the TALEN target site for the test samples with either TALEN binding pair SEQ ID NO:34 and SEQ ID NO:93 (approximately 7%) or SEQ ID NO:35 and SEQ ID NO:94 (approximately 16%) was significantly higher than the controls for these sites. In contrast, the percent integration of the dsDNA fragment into the TALEN target site for the test samples with either TALEN binding pair SEQ ID NO:295 and SEQ ID NO:296(approximately 2%) or SEQ ID NO:297 and SEQ ID NO:298 (approximately 0%) was not significantly different than the controls for these sites (FIG. 4). The DNA methylation for each of these specific TALEN target sites is presented in FIG. 4. The two TALEN target sites with TALEN binding pair SEQ ID NO:34 and SEQ ID NO:93 or TALEN binding pair SEQ ID NO:35 and SEQ ID NO:94 are located in relatively unmethylated regions. The two TALEN target sites with TALEN binding pair SEQ ID NO:295 and SEQ ID NO:296 or TALEN binding pair SEQ ID NO:297 and SEQ ID NO:298 are located in methylated regions (FIG. 4). These data illustrate that selecting TALEN target sites within genomic regions lacking DNA methylation increases the integration frequency of dsDNA into the TALEN target site.

### Example 4: TALEN Target Site Selection in Soy

The genomic sequence of Glycine max (germplasm A3555) was screened as detailed in Example 1 to identify optimal sites for genome modification, specifically to select TALEN target sites. From this analysis, 14 genomic regions were identified to contain TALEN target sites. For each genomic region, there were from one to 5 individual TALEN target sites identified for a total of 39 TALEN target sites (see Table 2). For each TALEN target site, the SEQ ID NO: corresponding each 5′-and 3′-TALEN binding site is presented in Table 2.

As one representative example of the target site selection process in soy, a genomic region on soy chromosome 2 (CR02), represented by SEQ ID NO:257, was chosen initially as being within a favorable haplotype window associated with a transgene insertion event with a positive agronomic trait for insect resistance. Although this genomic region was intergenic, after additional analysis no specific site was identified as a TALEN target site that met all of the selection criteria detailed in Example 1. Therefore, the region was reanalyzed with relaxed criteria for redundancy score and DNA methylation profile to select a TALEN target site.

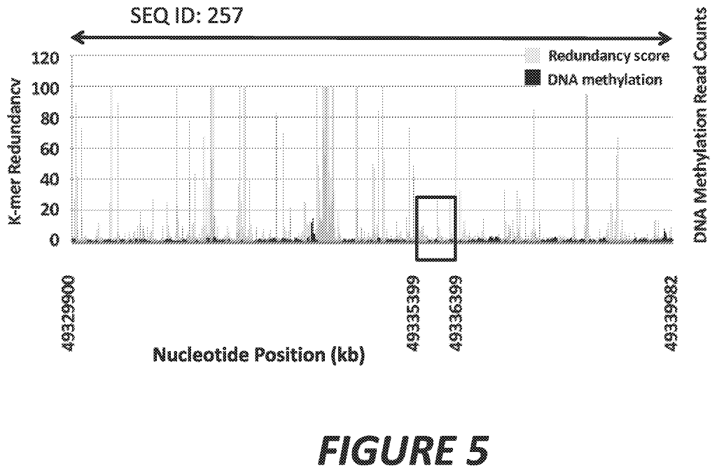

Redundancy scores for the selected genomic region were calculated as detailed in Example 1 using an 18 bp k-mer scanning window. The resulting k-mer redundancy scores were mapped to the haplotype window, and their distribution was scanned to identify genomic regions of at least 1 Kb long that had a total redundancy score of≤30%. No region of SEQ ID NO:257 met this selection criteria (FIG. 5). Therefore, the region was reanalyzed, and the preference for a 1 Kb region was relaxed to identify regions of at least 100 bp that had a total redundancy score of≤30%. Within the heterogeneous 9.6 kb region, several 100 bp stretches with total redundancy scores≤30% were interspersed between peaks of high sequence redundancy. These short, low redundancy sections formed the population from which TALEN target sites were selected.

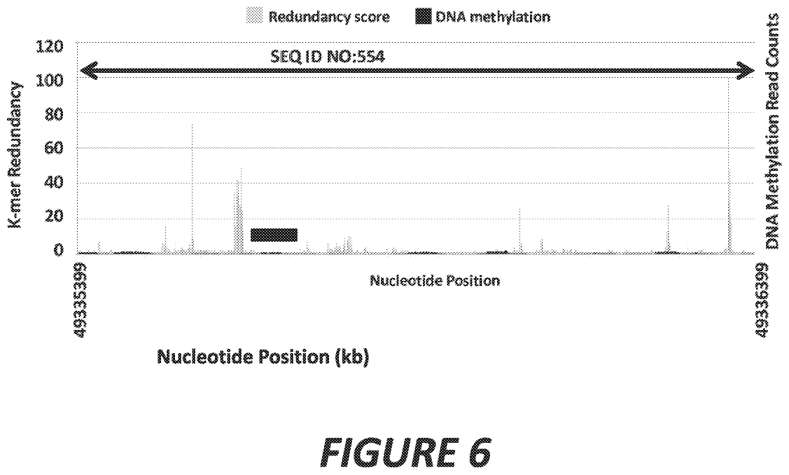

The low redundancy stretches were further analyzed to identify regions that lacked DNA methylation. DNA methylation was determined for the soy genome essentially as described in Example 3, and DNA methylation reads were mapped across the 9.6 kb region of SEQ ID NO:257. Similar to the total redundancy scores, the DNA methylation reads were heterogeneously distributed across the region (FIG. 5). Mapping the DNA methylation profiles across the population of short, low redundancy regions identified a 1 kb region (FIG. 6, SEQ ID NO:554) with one or more 150 bp regions meeting the relaxed criteria for redundancy and DNA methylation. Three TALEN target sites within SEQ ID NO:554 were selected, corresponding to 5′-and 3′-TALEN binding sites (a) SEQ ID NO:233 and SEQ ID NO:234; (b) SEQ ID NO:235 and SEQ ID NO:236; and (c) SEQ ID NO:237 and SEQ ID NO:238 (Table 2); with the relative position of all three TALEN binding sites illustrated by the thick horizontal line in FIG. 6.

For each of the TALEN target sites identified, TALEN pairs were engineered and TALEN activity was determined as described in Example 3, except using soy protoplasts for the assay. TALEN activity for each of the TALEN target sites assessed with the soy protoplast assay was determined by ddPCR, or by standard PCR with amplicon analysis by agarose gel electrophoresis (Table 2). In Table 2, if either ddPCR or the standard PCR was positive, then the TALEN activity was scored as active. If both assay results were negative for a particular TALEN target site, then the TALEN activity was scored as not active.

TABLE 2
TALEN activity measured by DNA integration at
individual soy TALEN target sites.

| Genomic | 5′ TALEN | 3′ TALEN binding | TALEN activity |
|---|---|---|---|
| region | binding site | site | for DNA integration |
| SEQ ID NO: | SEQ ID NO: | SEQ ID NO: | (active/not active) |
| 251 | 173 | 174 | active |
| 251 | 175 | 176 | active |
| 251 | 177 | 178 | active |
| 251 | 179 | 180 | not active |
| 252 | 181 | 182 | active |
| 252 | 183 | 184 | not active |
| 252 | 185 | 186 | not active |
| 252 | 187 | 188 | active |
| 252 | 189 | 190 | not active |
| 253 | 191 | 192 | active |
| 253 | 193 | 194 | not active |
| 253 | 195 | 196 | not active |
| 260 | 197 | 198 | active |
| 260 | 199 | 200 | not active |
| 260 | 201 | 202 | active |
| 262 | 203 | 204 | not active |
| 262 | 205 | 206 | not active |
| 262 | 207 | 208 | not active |
| 266 | 209 | 210 | active |
| 274 | 211 | 212 | not active |
| 274 | 213 | 214 | active |
| 275 | 215 | 216 | active |
| 275 | 217 | 218 | active |
| 281 | 219 | 220 | active |
| 255 | 221 | 222 | active |
| 255 | 223 | 224 | active |
| 255 | 225 | 226 | active |
| 256 | 227 | 228 | active |
| 256 | 229 | 230 | active |
| 256 | 231 | 232 | active |
| 257 | 233 | 234 | active |
| 257 | 235 | 236 | active |
| 257 | 237 | 238 | active |
| 258 | 239 | 240 | active |
| 258 | 241 | 242 | active |
| 258 | 243 | 244 | active |
| 259 | 245 | 246 | not active |
| 259 | 247 | 248 | active |
| 259 | 249 | 250 | active |

To further confirm the TALEN activity, a subset of the protoplast assay samples were reevaluated for successful integration of the dsDNA fragment into TALEN target sites by standard PCR using multiple primer sets (Table 3). For each PCR primer set, one primer was to sequence flanking the DSB of the TALEN target site, and one primer (SEQ ID NO:3) was specific to the dsDNA fragment integrated into the DSB. The PCR amplicons were separated using standard agarose gel electrophoresis, and the size of each amplicon was confirmed by comparison to a molecular weight marker. DNA samples from protoplast assay negative controls lacked PCR amplicons. A band of the expected size was detected for all three TALEN target sites for SEQ ID NO:257 (Sample #10, 11, and 12, Table 3). Of the 21 samples retested using standard PCR, TALEN activity was consistent between the two assays, standard PCR and ddPCR. These data indicate the utility in the selection process to identify target sites for genome modification in soy.

TABLE 3
PCR confirmation of site directed integration of dsDNA at
TALEN target sites in the soy genome.

| | 5′/3′ TALEN | | Size (bp) of | Expected band |
|---|---|---|---|---|
| | binding site | Primer pair | Expected | Amplified |
| Sample # | SEQ ID NO: | SEQ ID NO: | band | Yes or No |
| 1 | 199/200 | 290/3 | 750 | No |
| 2 | 211/212 | 292/3 | 1100 | No |
| 3 | 191/192 | 289/3 | 500 | Yes |
| 4 | 221/222 | 283/3 | 450 | Yes |
| 5 | 223/224 | 283/3 | 400 | Yes |
| 6 | 225/226 | 283/3 | 500 | Yes |
| 7 | 227/228 | 284/3 | 400 | Yes |
| 8 | 229/230 | 284/3 | 200 | Yes |
| 9 | 231/232 | 284/3 | 330 | Yes |
| 10 | 233/234 | 285/3 | 300 | Yes |
| 11 | 235/236 | 285/3 | 270 | Yes |
| 12 | 237/238 | 285/3 | 220 | Yes |
| 13 | 239/240 | 286/3 | 550 | Yes |
| 14 | 241/242 | 286/3 | 460 | Yes |
| 15 | 243/244 | 286/3 | 460 | Yes |
| 16 | 245/246 | 287/3 | 700 | No |
| 17 | 247/248 | 287/3 | 650 | Yes |
| 18 | 249/250 | 287/3 | 550 | Yes |
| 19 | 209/210 | 291/3 | 400 | Yes |
| 20 | 197/198 | 290/3 | 500 | No |
| 21 | 185/186 | 288/3 | 780 | No |

### Example 5: Analysis of Site Selection Methods

To evaluate the target site selection process, a population of RO transgenic corn events containing a transgene conferring herbicide tolerance were selected, and the genomic site of the randomly integrated transgene was determined using standard molecular biology and sequencing methods. Only the events with the transgene localized to intergenic regions were selected for the analysis. Additionally, the R0 events received application of herbicide in a greenhouse and were evaluated for herbicide tolerance as measured by the percentage of injury after herbicide application. Only the events with R0 injury scores within the range from 5 (low) to 30 (high) were included in the analysis. The size of the selected R0 population was 319 events. In this analysis, the integration coordinates of the randomly generated events were mapped, and these coordinates were evaluated to identify the number of events which would have been selected by the of site selection process as detailed in Example 1. Through this evaluation, the genomic location of randomly integrated transgene cassette identified 57 events which were within loci which would have been selected by the site selection process as described in Example 1.

A separate subset of randomly generated events were fertilized to set seed (inbred/hybrid) and the R1 were evaluated for herbicide tolerance in field trials. Analysis was completed for 24 randomly generated events that passed the R0 herbicide tolerance test but failed the field test. R1 plants from 24 events failed the field test with herbicide injury scores of≥30. Review of the 24 genomic integration sites of the R1 events that failed the field trial indicated that none of the sties of transgene integration met the criteria for target site selection detailed in Example 1. This analysis indicates the effectiveness of the target site selection process to identify genomic sequences for site-specific transgene integration that are optimal for agronomic performance of the transgenic trait.

### Example 6: Target Site Selection for Corn B Chromosome Sequence

The target site selection process essentially as detailed in Example 1 was repeated on a unique assembly of corn B chromosome sequence. The site selection criteria applied during the analysis included analysis of genic/intergenic regions, sequence redundancy, repeat analysis, DNA methhylation profile, and sRNA hotspot determination. From this analysis, 252 sequences (SEQ ID NO:300-551) were identified as potential loci for genome modification.

### Example 7: Validating TALEN Activity via Transgene Integration Into Target Sites in Corn

Nine genomic regions from the LH244 corn germplasm were selected for further testing of site specific genome modification by TALENs. Specifically, TALENs were engineered to introduce DSBs at loci within these sites to facilitate site-specific integration of a transgene cassette. The selected genomic regions are represented by SEQ IDs 123, 124, 127, 128, 132, 133, 137, 138 and 139. For each region, one TALEN target site was selected for testing TALEN activity. A TALEN target site included a 5′-TALEN binding site, a spacer sequence, and a 3′TALEN binding site. Each of the TALEN binding sites were 15 to 24 bp long, and the spacer sequence was 18 to 25 bp long. Within the spacer sequence is the site of the DSB induced by the TALEN, and the site of incorporation of the transgene cassette. The SEQ ID NOs for the 5′-TALEN binding site and 3′-TALEN binding site corresponding to each of the nine TALEN target sites are represented in Table 4. An expression cassette encoding CP4-EPSPS, which confers tolerance to the herbicide glyphosate, was chosen as the transgene for site-specific integration at the selected loci. The CP4-EPSPS transgene was flanked by homology arms (HA) to promote HR-mediated integration.

Individual T-DNA vectors comprising the transgene and TALEN pairs were generated for each locus. Each vector comprised two right borders (RBs) that flanked three expression cassettes: an expression cassette encoding the gene (CP4-EPSPS) positioned between a left homology arm and a right homology arm; and two expression cassettes cach encoding half of a TALEN pair created for a specific target site. TALENs were obtained from Life Technologies.

Approximately 3,800 to 6,800 immature corn embryos were co-cultured with Agrobacterium containing the vectors for 3 days, then moved to callus-induction medium containing 0.1 mM glyphosate as a selection agent. Approximately 30 to 250 regenerated plants were selected for each construct, transferred to plugs, and grown in a greenhouse.

To confirm TALEN-mediated site-directed integration, genomic DNA was isolated from selected RO plants and flank PCR assays were carried out to identify individual plants comprising CP4-EPSPS cassette insertions at the TALEN target sites. PCR primers were designed such that a product was only produced when the CP4-EPSPS cassette inserted into the selected target region of the corn genome. One PCR primer was designed to bind to genomic DNA flanking the targeted region, and one PCR primer was designed to bind to a sequence within the CP4-EPSPS cassette. Two sets of PCR primers were used, one positioned on the 5′ end of the CP4-EPSPS cassette and one positioned on the 3′ end of the CP4-EPSPS cassette. Following PCR, the PCR products were resolved on agarose gels to identify plants with the correct sized bands for both the 5′ end (5′ flank) and the 3′ end (3′ flank) of the CP4-EPSPS cassette. As tabulated in Table 4, TALEN activity and transgene insertion was observed in target loci 128, 133, 137, 138 and 139. This confirms that among the preselected corn genomic loci, several can be targeted for precise in planta genome modifications including transgene integration.

TABLE 4
TALEN activity measured by transgene integration at
individual LH244 TALEN target sites.

| Genomic | | 3′ TALEN | | # of |
|---|---|---|---|---|
| region | 5′ TALEN | binding site | | events positive |
| SEQ ID | binding site | SEQ ID | # of events | for Flank PCR |
| NO: | SEQ ID NO: | NO: | tested | (5′ or 3′ Flank) |
| 123 | 8 | 67 | 34 | 0 |
| 124 | 12 | 71 | 28 | 0 |
| 127 | 21 | 80 | 138 | 0 |
| 128 | 27 | 86 | 120 | 7 |
| 132 | 42 | 101 | 255 | 0 |
| 133 | 44 | 103 | 80 | 6 |
| 137 | 56 | 115 | 90 | 6 |
| 138 | 59 | 118 | 59 | 16 |
| 139 | 63 | 122 | 169 | 3 |

### Example 8: CRISPR-Cas9 Target Selection and Site-Specific DNA Integration in Corn protoplasts

After confirming that TALENs can be successfully used to introduce site specific modifications in selected genomic loci in protoplasts (Example 3), a similar assay was carried out to test for CRISPR-Cas9 mediated genome modifications at a pre-selected locus. A genomic sequence of 3.5 kb (SEQ ID NO:130) was chosen as a region to identify a target site, with the 1.6 kb sequence (SEQ ID NO:299) selected as the optimal region, thus avoiding the endogenous gene plus 2 kb buffer (Zm.B73 CR01 coordinates 287444 kb to 287445.9 kb), as illustrated in FIG. 2 and described in Example 3. This sequence was further analyzed and an optimal Cas9 guide RNA target site (SEQ ID NO:555) with a 3′ TGG PAM sequence was identified. A portion of the optimal region (SEQ ID NO:299) comprising sequences flanking the target site (SEQ ID NO:555) and TGG PAM sequence is also disclosed here as SEQ ID NO:556.

The assay used to evaluate CRISPR-Cas9 activity was integration of a blunt-end, double-stranded DNA (dsDNA) donor into the DSB created by the Cas9 nuclease at the specific target site. The CRISPR/Cas9 nuclease from Streptococcus pyogenes was chosen as the nuclease system. Two expression vectors were generated. One comprised an expression cassette for the Cas9 nuclease and the other comprised an expression cassette for the single-guide RNA designed to target SEQ ID NO:555. The expression vectors were introduced into isolated corn leaf protoplasts essentially as described in Example 3 and in patent application publication WO2015131101, with minor modifications. Briefly, complementary ssDNA oligonucleotides (SEQ ID NO:1 and SEQ ID NO:2) were pre-annealed to form a blunt-end, double-stranded DNA (dsDNA) donor. Transformations of isolated corn leaf protoplasts were performed using standard PEG-protocol, with 50 pmoles of the dsDNA fragment, and the two expression vectors (0.1 pmole cach) (Table 5, Test). Protoplast samples transformed in the presence of the dsDNA donor and the Cas9 plasmid but lacking the guide RNA plasmid; or transformed with the dsDNA fragment and the guide RNA plasmid but lacking the Cas9plasmid were used as negative controls (Table 5, Control 1 and Control 2). The corn protoplasts were harvested 48 hours after transformation, and the genomic DNA was assayed for integration of the dsDNA donor. Integration of the dsDNA donor into the genomic DNA was detected by standard PCR and agarose gel electrophoresis to assess PCR amplicons. The dsDNA donor may be integrated in either a 5′ or 3′ orientation with respect to the 5′-and 3′-ends of the DSB. Therefore, at least two PCR primer sets were run for the target site where the primer sets contained a primer specific to the dsDNA donor (SEQ ID NO:3), and a primer specific to either the 5′ side or the 3′ side of the DSB. Transformation efficiency of protoplasts was calculated using a control plasmid expressing green fluorescent protein using the method described in patent application publication WO2015131101.

PCR amplicons were separated using standard agarose gel electrophoresis, and the size of each amplicon was confirmed by comparison to a molecular weight marker. As shown in Table 5, a band of the expected size was detected in protoplasts expressing the Cas9, guide RNA and the dsDNA donor (Test) indicating site-directed integration of donor dsDNA at the target site following Cas9-mediated genomic DNA cleavage. DNA samples from protoplasts transformed with the negative controls lacked PCR amplicons (Control 1 and Control 2). To further confirm dsDNA donor integration, the gel-separated PCR amplicons from the Test samples were isolated, cloned via Zero blunt end Topo cloning (Life technologies) and sequenced. Sequence analysis of the target-donor junctions identified two independent integration events. Both events resulted from NHEJ-mediated donor dsDNA integration at the expected Cas9 cleavage site within the targeted genomic region. The results presented here demonstrate that the selected genomic locus is amenable to CRISPR-Cas9 mediated cleavage and site-specific integration of a donor dsDNA construct.

TABLE 5
Cas9 activity measured by DNA integration at
a specific target site in LH244 protoplasts.

| | Genomic | Target | | | | Expected |
|---|---|---|---|---|---|---|
| | region | site | | | Donor | band |
| | selected for | SEQ ID | Cas 9 | sg | oligonu- | amplified |
| Assay | targeting | NO: | Nuclease | RNA | cleotide | (Yes/No) |
| Test | SEQ ID | SEQ ID | + | + | + | Yes |
| | 130; 299 | 555 | | | | |
| Control- | SEQ ID | SEQ ID | + | − | + | No |
| 1 | 130; 299 | 555 | | | | |
| Control- | SEQ ID | SEQ ID | − | + | + | No |
| 2 | 130; 299 | 555 | | | | |

### Example 9: CRISPR-Cas9 Mediated Site-Specific DNA Integration at a Selected Target Site in Corn Embryos

After confirming that CRISPR-Cas9 could be successfully used to introduce site specific modifications in a selected genomic locus in a protoplast assay system, a similar assay was carried out in corn immature embryos. As described in Example 6, an optimal Cas9 gRNA target site (SEQ ID NO:555) was identified within a selected genomic locus (SEQ ID NOs:130 and 299).

The assay used to evaluate CRISPR-Cas9 activity was integration of a blunt-end, double-stranded DNA (dsDNA) donor oligo into the DSB created by the Cas9 nuclease at the selected target site. The CRISPR/Cas9 nuclease from Streptococcus pyogenes was expressed in E.coli and purified. The complementary ssDNA oligonucleotides (SEQ ID NO:1 and SEQ ID NO:2) were pre-annealed to form a blunt-end, double-stranded DNA (dsDNA) donor. The purified Cas9 protein, in-vitro synthesized guide RNA and the dsDNA donor were co-delivered into LH244 immature corn embryos via biolistics. Genomic DNA was extracted from the bombarded embryos after 48 hours and assayed for integration of the dsDNA donor. DNA extracted from untransformed embryos was used as a control.

As described in Example 8, integration of the dsDNA donor into the genomic DNA was detected by standard PCR and agarose gel electrophoresis to assess PCR amplicons. The dsDNA donor may be integrated in either a 5′ or 3′ orientation with respect to the 5′-and 3′-ends of the DSB. Therefore, at least two PCR primer sets were run for the target site where the primer sets contained a primer specific to the dsDNA donor (SEQ ID NO:3), and a primer specific to either the 5′ side or the 3′ side of the DSB. PCR amplicons were separated using standard agarose gel electrophoresis, and the size of each amplicon was confirmed by comparison to a molecular weight marker.

As shown in Table 6, a band of the expected size was detected in samples prepared from embryos bombarded with the Cas9, the guide RNA, and the dsDNA donor indicating site-directed integration of donor dsDNA at the target site following Cas9 mediated genomic DNA cleavage (Table 6, Test). No PCR product was detected in samples from untransformed tissue (Table 6, Control). To further confirm donor oligo integration, the gel-separated PCR amplicons were isolated, cloned via Zero blunt end Topo cloning (Life technologies) and sequenced. Sequence analysis of the target-donor junctions identified one independent integration event. This event resulted from NHEJ-mediated donor dsDNA integration at the expected Cas9 cleavage site within the targeted genomic region. The results presented here demonstrate that the selected genomic locus is amenable to CRISPR-Cas9mediated cleavage and site-specific integration of a donor dsDNA construct in corn embryogenic tissue. Taken together, results from Example 3, Example 7, Example 8 and Example 9 demonstrate that a locus selected according to criteria described in Example 1 can be precisely and reproducibly modified by multiple sequence specific endo-nucleases in multiple cell types for integration of donor DNA.

TABLE 6
Cas9 activity measured by DNA integration at
a specific target site in LH244 embryogenic tissue.

| | Genomic | | | | | Expected |
|---|---|---|---|---|---|---|
| | region | | | | Donor | band |
| | selected for | Target | | sg | oligonu- | amplified |
| Assay | targeting | site | Nuclease | RNA | cleotide | (Yes/No) |
| Test | SEQ ID | SEQ ID | + | + | + | Yes |
| | 130; 299 | 555 | | | | |
| Control | SEQ ID | SEQ ID | − | − | − | No |
| | 130; 299 | 555 | | | | |

What is claimed is:

1. A recombinant sequence comprising a non-genic plant genomic sequence and a DNA of interest, wherein the DNA of interest is integrated into a target site in the non-genic plant genomic sequence, and wherein the target site is located in a haplotype window associated with a neutral to positive impact on one or more agronomic traits, wherein the target site is further located at genetic distance greater than 1 cM of a haplotype window that is associated with a negative impact on one or more agronomic traits and wherein the non-genic plant genomic sequence is a corn genomic sequence selected from the group consisting of SEQ ID NOs: 123-172, 294, 299-551, 555 and 556 or a soybean genomic sequence selected from the group consisting of SEQ ID NOs:252-282 and 554.

2. The recombinant sequence of claim 1, wherein the target site is located less than 1000 bp of low genetic diversity, wherein low genetic diversity is defined as having from one to ten distinguishable haplotypes across all germplasm in the intended heterotic group, the intended maturity group, or the intended heterotic and maturity group.

3. The recombinant sequence of claim 1, wherein the haplotype window is between 40 base pairs and the full length of the chromosome, with at least 99% sequence similarity across targeted germplasm and contains two or fewer indels of transposon size (˜3kb).

4. The recombinant sequence of claim 1, wherein the haplotype window is defined by genetic distance and wherein the genetic distance is 0.1 cM, 0.2 cM, 0.3 cM, 0.4 cM, 0.5 CM, 0.6 cM, 0.7 cM, 0.8 CM, 0.9 cM, 1 cM, 1.1 cM, 1.2 cM, 1.3 cM, 1.4 cM, 1.5 CM, 1.6 cM, 1.7 cM, 1.8 CM, 1.9 cM, 2 cM, 2.1 cM, 2.2 cM, 2.3 cM, 2.4 cM, 2.5 CM, 2.6 cM, 2.7 CM, 2.8 CM, 2.9 cM, 3 cM, 3.1 cM, 3.2 cM, 3.3 cM, 3.4 cM, 3.5 CM, 3.6 cM, 3.7 cM, 3.8 CM, 3.9 cM, 4 cM, 4.1 cM, 4.2 cM, 4.3 cM, 4.4 cM, 4.5 CM, 4.6 cM, 4.7 cM, 4.8 cM, 4.9 cM, or 5 cM.

5. The recombinant sequence of claim 1, wherein the agronomic trait is one or more selected from the group consisting of: yield, ear relative maturity, ear height, ear number, increased ear size, grain moisture, increased ear dry weight per plant, increased number of kernels per ear, increased weight per kernel, increased number of kernels per plant, decreased ear void, extended grain fill period, test weight, pod number, number of seed per pod, pod position on the plant, number of internodes, incidence of pod shatter, grain size, decreased days from planting to maturity, increased stalk size, increased number of leaves, increased plant height growth rate in vegetative stage, plant architecture, resistance to lodging, percent seed germination, seedling vigor, juvenile traits, efficiency of germination (including germination in stressed conditions), growth rate (including growth rate in stressed conditions), increased number of root branches, increased total root length, efficiency of nodulation and nitrogen fixation, enhanced nitrogen use efficiency, increased water use efficiency as compared to a control plant, efficiency of nutrient assimilation, resistance to biotic and abiotic stress, carbon assimilation, physiology, enhanced disease or pest resistance, or environmental or chemical tolerance, enhanced cold tolerance, nutritional enhancement, enhanced seed protein, enhanced seed starch, enhanced seed oil, plant height, enhanced plant morphology, growth and development, and stay green rating.

6.-8. (canceled).

9. The recombinant sequence of claim 1, wherein the target site comprises at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 300, at least 400, at least 500, at least 600, at least 700, at least 800, at least 900, or at least 1000 nucleotides.

10. The recombinant sequence of claim 1, wherein the target site comprises one or more, two or more, three or more, four or more, five or more, six or seven of the criteria selected from the group consisting of:
i. the target site is located greater than 2 kb from a 5′ or a 3′ end of a gene in the plant genome;
ii. the target site is located more than 1 kb from a 5′ or a 3′ end of a repeat region in the plant genome, and wherein the repeat region is at least 2 kb in length;
iii. the target site is located more than 1 kb from a 5′ or a 3′ end of a repressive chromatin mark in the plant genome;
iv. the target site is located more than 200 bases from a small RNA (sRNA) hotspot in the plant genome, and wherein the sRNA hotspot is a sequence from 0.2 to 1 kb in length;
v. the target site is within a region of the plant genome of low DNA methlyation;
vi. the target site is not within a region of the plant genome associated with at least one DNA methylation read containing an MspJi motif or a LpnPI motif;
vii. the target site is within a region of the plant genome that exhibits a total k-mer redundancy score of less than or equal to 30%.

11. The recombinant sequence of claim 1, wherein the DNA of interest comprises a gene expression cassette comprising a sequence selected from an insecticidal resistance gene, a herbicide tolerance gene, a nitrogen use efficiency gene, a water use efficiency gene, a nutritional quality gene, a DNA binding gene, a selectable marker gene, and any combination thereof.

12. A method of making a transgenic plant cell comprising a DNA of interest targeted to at least one non-genic plant genomic sequence, the method comprising:
i. selecting a target site located within a haplotype window associated with a neutral to positive impact on one or more agronomic traits;
ii. introducing a site-specific genome modification enzyme into a plant cell, wherein the site-specific genome modification enzyme cleaves the target site in the non-genic plant genomic sequence;
iii. introducing a DNA of interest;
iv. targeting the DNA of interest to the target site, wherein the cleavage of the target site facilitates integration of the DNA of interest into the non-genic plant genomic sequence; and
v. selecting transgenic cells comprising the DNA of interest integrated into the non-genic plant genomic sequence, wherein the non-genic plant genomic sequence is a corn genomic sequence selected from the group consisting of SEQ ID NOs: 123-172, 294, 299-551, 555 and 556 or a soybean genomic sequence selected from the group consisting of SEQ ID NOs:252-282 and 554.

13. The method of claim 12, wherein the target site is further located at a genetic distance of greater than 10 cM of a haplotype window that is associated with a negative impact on one or more agronomic traits.

14. The method of claim 12, wherein the genetic distance of the haplotype window is 0.1 cM, 0.2 cM, 0.3 cM, 0.4 cM, 0.5 CM, 0.6 cM, 0.7 CM, 0.8 CM, 0.9 cM, 1 cM, 1.1 cM, 1.2 cM, 1.3 cM, 1.4 cM, 1.5 CM, 1.6 cM, 1.7 CM, 1.8 cM, 1.9 cM, 2 CM, 2.1 cM, 2.2 cM, 2.3 cM, 2.4 cM, 2.5 CM, 2.6 cM, 2.7 cM, 2.8 CM, 2.9 cM, 3 CM, 3.1 cM, 3.2 cM, 3.3 cM, 3.4 cM, 3.5 cM, 3.6 cM, 3.7 cM, 3.8 cM, 3.9 cM, 4 cM, 4.1 cM, 4.2 cM, 4.3 cM, 4.4 cM, 4.5 CM, 4.6 cM, 4.7 cM, 4.8 cM, 4.9 cM, or 5 cM.

15. The method of claim 12, wherein the agronomic trait is one or more selected from the group consisting of: yield, ear relative maturity, ear height, ear number, increased ear size, grain moisture, increased ear dry weight per plant, increased number of kernels per ear, increased weight per kernel, increased number of kernels per plant, decreased ear void, extended grain fill period, test weight, pod number, number of seed per pod, pod position on the plant, number of internodes, incidence of pod shatter, grain size, decreased days from planting to maturity, increased stalk size, increased number of leaves, increased plant height growth rate in vegetative stage, plant architecture, resistance to lodging, percent seed germination, seedling vigor, juvenile traits, efficiency of germination (including germination in stressed conditions), growth rate (including growth rate in stressed conditions), increased number of root branches, increased total root length, efficiency of nodulation and nitrogen fixation, enhanced nitrogen use efficiency, increased water use efficiency as compared to a control plant, efficiency of nutrient assimilation, resistance to biotic and abiotic stress, carbon assimilation, physiology, enhanced disease or pest resistance, or environmental or chemical tolerance, enhanced cold tolerance, nutritional enhancement, enhanced seed protein, enhanced seed starch, enhanced seed oil, plant height, enhanced plant morphology, growth and development, and stay green rating.

16.-18. (canceled)

19. The method of claim 12, wherein the target site comprises one or more, two or more, three or more, four or more, five or more, six or seven of the criteria selected from the group consisting of:
i. the target site is located greater than 2 kb from a 5′ or a 3′ end of a gene in the plant genome;
ii. the target site is located more than 1 kb from a 5′ or a 3′ end of a repeat region in the plant genome, and wherein the repeat region is at least 2 kb in length;
iii. the target site is located more than 1 kb from a 5′ or a 3′ end of a repressive chromatin mark in the plant genome;
iv. the target site is located more than 200 bases from a small RNA (sRNA) hotspot in the plant genome, and wherein the sRNA hotspot is a sequence from 0.2 to 1 kb in length;
v. the target site is within a region of the plant genome of low a DNA methylation;
vi. the target site is not within a region of the plant genome associated with at 20 least one DNA methylation read sequence containing an MspJi motif or a LpnPI motif; and
vii. the target site is within a region of the plant genome that exhibits a total k-mer redundancy score of less than or equal to 30%.

20. The method of claim 12, wherein the target site comprises at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 300, at least 400, at least 500, at least 600, at least 700, at least 800, at least 900, or at least 1000 nucleotides.

21. The method of claim 12, wherein said DNA of interest comprises a gene expression cassette comprising a sequence selected from an insecticidal resistance gene, a herbicide tolerance gene, a nitrogen use efficiency gene, a water use efficiency gene, a nutritional quality gene, a DNA binding gene, a selectable marker gene, and any combination thereof.

22. The method of claim 12, wherein the site-specific genome modification enzyme is selected from an endonuclease, a recombinase, a transposase, and any combination thereof.

23. The method of claim 22, wherein the endonuclease is selected from a meganuclease, a zinc finger nuclease, a transcription activator-like effector nuclease (TALEN), a Cas9 nuclease, and a Cpf1 nuclease.

24. The method of claim 22, wherein the recombinase is a tyrosine recombinase attached to a DNA recognition motif, or a serine recombinase attached to a DNA recognition motif.

25. The method of claim 24, wherein the tyrosine recombinase attached to a DNA recognition motif is selected from the group consisting of a Cre recombinase, a Flp recombinase, and a Tnp 1 recombinase.

26. The method of claim 24, wherein the serine recombinase attached to a DNA recognition motif is selected from the group consisting of a PhiC31 integrase, an R4 integrase, and a TP-901 integrase.

27. The method of claim 22, wherein the transposase is a DNA transposase attached to a DNA binding domain.

28. The method of claim 23, wherein the transcription activator-like effector nuclease (TALEN) DNA binding site within the target site of com genomic sequence is selected from the SEQ ID NOs presented in Table 1.

29. The method of claim 23, wherein the transcription activator-like effector nuclease (TALEN) DNA binding site within the target site of soybean genomic sequence is selected from the SEQ ID NOs presented in Table 2.

30. The method of claim 12, wherein the DNA of interest is an exogenous sequence.

31. The method of claim 12, wherein the DNA of interest is integrated into the target site via a non-homologous end joining.

32. The method of claim 12, wherein the DNA of interest is integrated into the target site via a homologous recombination.

33. A plant, plant cell, or plant part comprising the recombinant sequence of claim 1.
